(19) United States
(12) Patent Application Publication
(10) Pub. No.: US 2009/0317497 A1
(43) Pub. Date: Dec. 24, 2009

(54) Compositions and methods for promoting brain and cardiovascular health, preventing and treating brain and cardiovascular disorders

(76) Inventor: Wen Hsien Chou, Kowloon (HK)

Correspondence Address: WILSON, SONSINI, GOODRICH & ROSATI, 650 PAGE MILL ROAD, PALO ALTO, CA, 94304-1050, US

(21) Appl. No.: 12/457,155

(22) Filed: Jun. 2, 2009

Related U.S. Application Data
(62) Division of application No. 11/788,376, filed on Apr. 18, 2007, now abandoned.

(51) Int. Cl. A61K 36/258; A61P 9/00

(52) U.S. Cl. 424/728

(57) ABSTRACT

Compositions, kits and methods are provided for promoting brain and cardiovascular health, preventing or treating neurological and vascular disorders. In one embodiment, the composition comprises Rhodiola rosea (root), Ginkgo biloba (leaf), Panax notoginseng (root), and Ligusticum chuanxiong (rhizome). The composition can be used as pharmaceuticals or nutraceuticals for promoting general brain health, maintaining a healthy brain, and all neurological disorders such as memory loss, ischemic stroke, neurodegenerative diseases (e.g., Huntington's disease, Alzheimers's disease, and amyotrophic lateral sclerosis) and vascular diseases such as arteriosclerosis, congestive heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like.

### CROSS-REFERENCE

This application claims the benefit of U.S. Provisional Application No. 60/793,956, filed Apr. 20, 2006, and is related to U.S. application Ser. No. 11/788,376, filed Apr. 18, 2007, of which both applications are incorporated herein by reference.

### FIELD OF THE INVENTION

The present invention is related to compositions and methods for promoting brain and cardiovascular health, and for preventing and treating brain and cardiovascular disorders, and more particularly related to methods and compositions for maintaining blood circulation and brain health, preventing and treating ischemic stroke, neurodegenerative diseases such as Huntington's disease, Alzheimers's disease, and amyotrophic lateral sclerosis, and vascular diseases such as arteriosclerosis, congestive heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like.

### BACKGROUND OF THE INVENTION

Brain plays the most important vital and central role in the body. Being the major part of the central nervous system, it controls many important activities in the body. Thoughts, memory, logic deduction and induction, mental, cognitive, and intellectual functions are all activities of the brain cells. Brain also controls the body's motor activities and detect sensory signals from sensory organs. Heart rate, respiration, and many other vital physiological activities are controlled by the brain. Brain cells are very sensitive cells and they are susceptible to permanent cell damage when there is poor blood circulation supplying oxygen and nutrients, such as in an ischemic stroke.

Stroke is the second leading cause of death and a significant cause of adult disability in the world. Eminent risk factors include cigarette smoking, hypertension and hyperlipidaemia. Stroke is an abrupt loss of brain function as a consequence of interference with the blood supply to the central nervous system (CNS). Acute stroke can be classified into two major categories—hemorrhage and ischemia. Hemorrhage refers to the rupture of a blood vessel present in the brain, thus leading to the leakage of blood into the brain cavity and subsequently causing damage to the brain. On the other hand, ischemia, which represents 80% of all stroke cases, causes damage to the brain by a reduction or total blockage of blood flow to parts of the brain, resulting in oxygen and glucose deficiency. In order to gain a deeper understanding of mechanisms underlying stroke, there are generally two animal models of cerebral ischemia that are employed in brain ischemic studies—global ischemia and focal ischemia. Global ischemia is the result of a systemic decrease in blood flow caused by a decrease in blood volume or low blood pressure, thus affecting the whole brain. On the contrary, focal ischemia affects only a part of the brain by means of blood vessel occlusion by natural causes such as thrombosis or embolism, leading to severe restriction or total blockage of blood flow to the brain.

Following cerebral ischemia, the regional cerebral blood flow (rCBF) drops below 10% of control values in the infarct core (the region proximal to the site of occlusion). Insufficient supplies of glucose and oxygen lowers adenosine-triphosphate (ATP) levels and seriously compromises metabolic processes that require energy. Ion channels that are ATP dependent are disrupted causing cell membrane depolarization causing electrical failure of the cell, resulting in the activation of excitatory amino acid (excitotoxicity) and neurotransmitter cascades. Excessive excitatory amino acids, such as glutamate, are released at the synapse in response to a considerable amount of injury to neurons. The surplus of glutamate activates the glutamate receptors which causes the opening of ion channels that permit sodium and calcium ions to enter the cell while allowing potassium ions to leave the cell.

Mitochondria are essential regulators of the brain cell response to ischemia as they play a role in ATP production, free radical production, control of apoptotic cell death as well as cellular calcium homeostasis. Mitochondria have a huge capacity to amass calcium. However, when intracellular calcium is maintained under normal dynamic physiological range, mitochondria do not sequester much calcium since the rate of calcium uptake and affinity for calcium are low. During calcium overload, when intracellular calcium surpasses 0.5 μM.

Mitochondria will begin sequestering significant quantities of calcium, which can trigger the opening of the mitochondrial permeability transition (MPT) pore opening. The opening of the MPT pore short-circuits the inner mitochondrial membrane to hydrogen ions (H⁺), causing a collapse of the Hmitochondria will begin sequestering significant quantities of calcium, which can trigger the opening of the mitochondrial permeability transition (MPT) pore opening. The opening of the MPT pore short-circuits the inner mitochondrial membrane to hydrogen ions (H⁺⁺ electrochemical gradient and ATP production. Furthermore, the pore opening releases calcium, uncouples oxidative phosphorylation resulting in a burst of reactive oxygen species (ROS) production as well as changes in mitochondrial permeability leading to the release of factors such as cytochrome c, Smac/Diablo, apoptosis inducing factor (AIF), heat shock protein 60, HtrA2/Omi and endonuclease G. AIF and endonuclease G have a proposed role in the induction of caspase-independent apoptotic changes in nuclei; cytochrome c participates directly in the activation of caspases while Smac/Diablo and HtrA2/Omi assist the activation of caspases by inhibiting proteins from the lap family (such as X-linked inhibitor of apoptosis (XIAP)), which are caspase inhibitors.

Mitochondrial dysfunction follows after cerebral ischemia since the drop in ATP content leads to the unsaturation of cytochrome oxidase at the terminus of the mitochondrial respiratory chain. This leads to a decrease in mitochondrial respiratory function. The interference of the mitochondrial electron transport system results in autooxidation of ubisemiquinone and flavoprotein to form superoxide (O₂⁻) radicals. Elevated levels of intracellular calcium also intensify ROS levels by the activation of phospholipase, while the conversion of xanthine or hypoxanthine and molecular oxygen generates hydrogen peroxide and oxygen radicals and urea. In addition, the autooxidation of catecholamines in the extracellular compartment as well as the presence of leukocytes generates a large fraction of ROS (Zhu et al., 2004). As a result, oxidative stress ensues due to the imbalance of cellular production of ROS and the incapacity of the cells to safeguard against them. The generation of these free radicals causes damage to cellular components such as lipids, especially polyunsaturated fatty acids, in which the double bonds within membranes allow ROS to remove hydrogen ions, a process known as lipid peroxidation. Furthermore, free radicals also destroy nucleic acids, such as deoxyribonucleic acid (DNA), peroxidation of proteins and carbohydrates, blood brain barrier break-down and microglial infiltration in the ischemic territory. In addition to inflammation and calcium overload, the damage of DNA by oxidative stress triggers apoptosis. The tumour suppressor p53 also participates in the cellular response to DNA damage, effecting cell cycle arrest, DNA repair and apoptosis. In areas of brain tissues less severely damaged (ischemia penumbra) as a result of retrograde perfusion by anastomosis from neighboring arteries, cerebral blood flow decreases to 20 to 40% of the normal conditions, cells are electrically silent, and yet they maintain a low level of metabolic activity and stability for a few hours. In this region, cell death is prolonged by hours or days since ATP levels are high enough for apoptosis to occur. Later on, inflammatory processes are triggered and immune mediated damage of neural tissues occurs.

Two major strategies are employed in the treatment of acute ischemic stroke—the vascular approach, in which the ischemic insult is limited by early reperfusion; and the cellular approach, whereby there is interference with the pathobiochemical cascade that results in ischemic neuronal damage. One necessary requirement for either of these approaches is the presence of functionally damaged but viable and potentially salvageable tissue. The time window for effective treatment is rather short for the vascular approach, while it is of longer duration for the cellular approach, especially for the anti-apoptotic and anti-inflammatory methods. Stroke therapies targeted at the ischemic core (whereby neurons die swiftly as a result of oxygen starvation) need to be fast and efficient in reversing the blockage of blood supply and being able to raise the blood flow above the critical threshold before cells become permanently damaged. On the other hand, the ischemic penumbra is deemed as the most promising target for stroke therapies as the therapeutic window is prolonged for several hours and because this area can be defined by functional neuroimaging modalities. Sufficient reperfusion before irreversible cell damage at the ischemic penumbra, as well as added neuroprotective agents aimed at different steps in the pathobiochemical cascade could help prevent or alleviate secondary ischemic cell damage (Heiss et al., 1999 Stroke 30:1486-1489). As such, many current neuroprotective strategies have been targeted at molecules that are able to intervene with apoptotic mechanisms in the penumbra where ATP levels are sufficient to allow energy-dependent apoptosis to take place. Scientists at Celgene Corporation (San Diego, Calif.) experimented with a c-Jun N-terminal kinase inhibitor and showed that the number of TdT-mediated dUTP nick end labeling (TUNEL)-positive cells, an indicator of the number of apoptotic cells was reduced (Harbeck, 2002 Drug Discov. Today 7:157-159). Transactivator domain (TAT)-fusion proteins that have transmembrane passage capabilities, such as fusion proteins containing the anti-apoptotic molecule Bcl-xL, showed a substantial reduction in cerebral infarction when administered intraperitoneally following focal transient ischemia. Another anti-apoptotic protein, Survivin, an inhibitor of caspase-1, promotes cell proliferation in vitro (Onteniente et al, 2003 Biochem. Pharmacol. 66:1643-1649).

Lorsatan, also known by its U.S brand name Cozaar®, received U.S Food and Drug Administration (FDA) approval in 1995. The first of a new class of antihypertensives, it works as a selective and competitive non-peptide Ang II receptor type AT₁ antagonist. It can be used alone or together with a diuretic, hydrochlorothiazide, which provides greater blood pressure-lowering effects. Losartan interacts reversibly at the AT₁ and AT₂ receptors of many tissues and has slow dissociation kinetics, having a 1000 times greater affinity for the AT₂ receptor than the AT₂ receptor (Lacy et al., 2003). Ang II is the main effector peptide of the rennin-angiotensin system in the brain which plays a crucial role in regulating blood pressure and fluid balance. The G-protein coupled receptors of Ang II are Ang II type 1 receptor (AT1R) and Ang II type 2 receptor (AT2R), sharing a limited homology of 34%. AT1R and AT2R have dissimilar functions and distributions in the brain. AT1R is predominant in the hypothalamus and brain stem, whereas AT2R is concentrated in the thalamus and specific brain stem nuclei. The majority of actions ascribed to Ang II are mediated by the AT1R, including vasoconstriction, aldosterone release, renal sodium reabsorption and cardiovascular hypertrophy. Other actions mediated by AT1R comprise of the enhancement of inflammation by means of macrophage activation and cell migration, smooth muscle cell proliferation and growth, as well as generation of oxygen free radicals. All these effects play a role in acute ischemic events. On the other hand, the function of AT2R is less well-defined. Stimulation of AT2R may enhance cell differentiation, mediate vasodilation via release of nitric oxide and cyclic guanosine monophosphate-mediated vasodilation, in which bradykinin may also be involved in effects of AT2R, as well as inhibiting cell proliferation and inflammatory responses (Schiffrin, 2002 Am. J. Med. 113:409-418).

A medical treatment approved by the FDA for acute ischemic stroke is tissue plasminogen activator (TPA), also known as alteplase, a thrombolytic agent that dissolved blood clots (Habeck, 2002). While alteplase is able to restore blood flow rapidly, the drug has to be administered within six hours after symptom starts and is linked to a rise in intracerebral hemorrhage incidences. Furthermore, permanent as well as transient re-occlusions related to increased mortality still arise after thrombolysis with alteplase (Lapchak and Araujo, 2003 Am. J. Cardiovasc. Drugs 3:87-94).

### SUMMARY OF THE INVENTION

The present invention provides novel compositions, kits and methods for pharmaceutical or nutraceutical use in an animal, preferably in a human.

In one aspect, compositions are provided, preferably for promoting brain health, preventing or treating brain disorders. The composition comprises at least 2 herbal ingredients selected from the group consisting of Rhodiola rosea, Ginkgo biloba, Panax notoginseng, and Ligusticum chuanxiong. Preferably, the composition comprises at least 3 herbal ingredients selected from the group consisting of Rhodiola rosea, Ginkgo biloba, Panax notoginseng, and Ligusticum chuanxiong. More preferably, the composition comprises Rhodiola rosea (root), Ginkgo biloba (leaf), Panax notoginseng (root), and Ligusticum chuanxiong (rhizome). A combination of these herbal ingredients may have synergistic effects on promoting blood circulation in the brain, prevention or treatment of memory loss, prevention or treatment of brain disorders such as ischemic stroke and neurodegenerative diseases (e.g., Huntington's disease, Alzheimers's disease, and amyotrophic lateral sclerosis) and vascular diseases such as arteriosclerosis, congestive heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like. The inventive compositions can be used as pharmaceuticals or nutraceuticals for promoting general brain health, maintaining a healthy brain, and all neurological and vascular disorders described above.

Another aspect of the invention provides compositions, preferably for promoting cardiovascular health, and preventing or treating cardiovascular diseases. It shall be understood that each composition otherwise described herein for promoting brain health may be alternatively applied for other indications related to promoting cardiovascular health. The combination of herbal ingredients provided herein may have synergistic effects on promoting blood circulation in the heart, prevention or treatment of heart and vascular diseases or disorders such as arteriosclerosis and atherosclerosis. In yet another embodiment, the inventive compositions can be used as pharmaceuticals or nutraceuticals for promoting general blood circulation throughout the body, maintaining a healthy brain, heart and other parts of a mammalian body to which circulation of blood is provided.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized.

FIG. 1 shows mortality rate of Wistar rats.

FIG. 2 shows neurological scores of Wistar Rats.

FIG. 3 shows infarct volume of Wistar rats.

FIG. 4 shows the Formulation A-treated rats (500 mg/kg/day) simple swim patterns in the Morris Water Maze Task when compared to the vehicle group.

FIG. 5 shows the Formulation A-treated group (500 mg/kg/day) had the lowest mean escape latency compared to all other treatment groups on day three and was lower than lingzhi and simvastatin on day 5.

FIG. 6 shows the Formulation A-treated group (500 mg/kg/day) had the lowest mean swim distance on day 2 compared to all other groups and was lower than the gingko, lingzhi and vehicle groups on days 3. This effect was not noted on day 5.

FIG. 7 shows the Formulation A-treated group (500 mg/kg/day) generally swam faster compared to other treatment groups.

FIG. 8 shows the Formulation A treated group swam more distance in zone four compared to ginkgo and lingzhi and slightly more than the vehicle group.

FIG. 9 shows the Formulation A-treated group (500 mg/kg/day) spent a longer time in zone 4 compared to gingko and lingzhi but in general all groups spent a similar time in zone 4. (Zone 4 is the region where the platform was originally located.)

FIG. 10 shows the Formulation A-treated group (250 mg/kg/day) did not affect the white blood cell count compared to the sham group.

FIG. 11 shows the Formulation A-treated groups (250 and 500 mg/kg/day) did not affect the red blood cell count compared to other treatment groups.

FIG. 12 shows the Formulation A-treated groups (250 and 500 mg/kg/day) did not affect the hemoglobin count compared to other treatment groups

FIG. 13 shows the Formulation A-treated group (250 mg/kg/day) did not affect MCV count in blood compared to the sham and vehicle groups.

FIG. 14 shows the Formulation A-treated group (250 mg/kg/day) did not affect MCH count in blood compared to the sham and vehicle groups.

FIG. 15 shows the Formulation A-treated groups (250 and 500 mg/kg/day) did not affect the hematocrit count compared to other treatment groups.

FIG. 16 shows the Formulation A-treated groups (250 and 500 mg/kg/day) did not affect the platelet count compared to other treatment groups.

FIG. 17 shows the Formulation A-treated group (250 mg/kg/day) did not affect MPV in blood compared to the sham and vehicle groups

FIG. 18 shows the Formulation A-treated group (250 mg/kg/day) did not affect MPV in blood compared to the sham and vehicle groups.

FIG. 19 shows the Formulation A-treated groups (500 mg/kg/day) did not affect the level of GOT in blood compared to the Sham group.

FIG. 20 shows the Formulation A-treated groups (250 and 500 mg/kg/day) did not affect the level of creatinine in blood compared to other treatment groups.

FIG. 21 shows both Formulation A-treated groups (250 and 500 mg/kg/day) did not affect the level of amylase in blood compared to other treatment groups.

FIG. 22 shows mortality rate of SHR strain.

FIG. 23 shows mortality rate of Wistar strain.

FIG. 24 shows Infarct Volume Assessment of SHR strain.

FIG. 25 shows Infarct Volume Assessment of Wistar strain.

FIG. 26 shows the neurological score for SHR strain after stroke.

FIG. 27 shows the neurological score for Wistar strain after stroke.

FIG. 28 shows HE staining of the number of capillaries for SHR strain.

FIG. 29 shows HE staining of the number of capillaries for Wistar strain.

FIG. 30 shows inhibition of Pyrogallol Red Bleaching by Hypochlorous Acid by HOCL.

FIG. 31 shows inhibition of ABTS Assay.

FIG. 32A shows AT2 receptor and the effects of studied drugs on the expression level of AT2 receptor.

FIG. 32B shows expression level of AT2 in each treatment group.

FIG. 33A shows effects of studied drugs on the expression level of Bax.

FIG. 33B shows expression level in each treatment group.

FIG. 34A shows effects of studied drugs on the expression level of Fas.

FIG. 34B shows expression level of Fas in each treatment group.

FIG. 35A shows the effects of studied drugs on the expression level of Bcl-xL and Bcl-xS.

FIG. 35B shows the expression level of Bcl-xL in each treatment group.

FIG. 35C shows the expression level of Bcl-xS in each treatment group.

FIG. 36 shows the Ratio of BcL-xL and BcL-xS in each treatment group.

FIG. 37 shows infarct volume of different treatment groups of Wistar rats.

FIG. 38 shows infarct volume of different treatment groups in SHR.

FIG. 39 shows Capillary count of different treatment groups after stroke in Wistar rats (n=3) (W.S: sham; W.S+BT: sham+Remembrance; W.Veh: vehicle; W.BT (250): Remembrance 250 mg; W.BT (500): Remembrance 500 mg; W.G: ginkgo; W.A: Aspirin; W.L: Losartan) †† p<0.01 when compared to sham group. ** p<0.01 when compared to vehicle group.

FIG. 40 shows the effect of different treatment on the expression level of AT2 receptor in Wistar rats (n=3). ** p<0.01 when compared with vehicle group. # p<0.05 when compared with sham group. § p<0.05 when compared with vehicle group.

FIG. 41 shows the effect of different treatment on the expression level of AT2 receptor in SHR (n=3).

FIG. 42 shows the effect of different treatment on the expression level of BAX in Wistar rats (n=3).

FIG. 43 shows the effect of different treatment on the expression level of BAX in SHR (n=3). ** p<0.01 when compared with vehicle group. * p<0.05 when compared to vehicle group. § p<0.05 when compared with vehicle group.

FIG. 44 shows the effect of different treatment on the expression level of FAS in Wistar rats (n=3). * p<0.05 when compared to vehicle group. # p<0.05 when compared with sham group. § p<0.05 when compared with vehicle group.

FIG. 45 shows the effect of different treatment on the expression level of FAS in SHR (n=3). * p<0.05 when compared to vehicle group. ** p<0.01 when compared with vehicle group. § p<0.05 when compared with vehicle group.

FIG. 46 shows the effect of different treatment on the expression level of Bcl-xL in Wistar rats (n=3). * p<0.05 when compared to vehicle group. ** p<0.01 when compared with vehicle group. § p<0.05 when compared with vehicle group.

FIG. 47 shows the effect of different treatment on the expression level of Bcl-xL in SHR (n=3). ** p<0.01 when compared with vehicle group. § p<6.05 when compared with vehicle group.

FIG. 48 shows the effect of different treatment on the expression level of Bcl-xS in Wistar rats (n=3). * p<0.05 when compared to vehicle group. § p<0.05 when compared with vehicle group.

FIG. 49 shows the effect of different treatment on the expression level of Bcl-xL in SHR (n=3). * p<0.05 when compared to vehicle group. ** p<0.01 when compared with vehicle group. § p<0.05 when compared with vehicle group.

FIG. 50 shows the effect of different treatment on the ratio of Bcl-xL and Bcl-xS in Wistar rats (n=3).

FIG. 51 shows the effect of different treatment on the ratio of Bcl-xL and Bcl-xS in SHR (n=3).

FIG. 52 shows the effect of different treatment on the expression level of AT1 in Wistar rats (n=3). ** p<0.01 when compared with vehicle group. § p<0.05 when compared with vehicle group.

FIG. 53 shows the effect of different treatment on the expression level of AT1 in SHR (n=3).

FIG. 54 shows the effect of different treatments on the expression level of eNOS in Wistar rats (n=3). ** p<0.01 when compared with vehicle group. # p<0.05 when compared with sham group. § p<0.05 when compared with vehicle group.

FIG. 55 shows oxidized DNA base products were analyzed and quantified by GC/MS and vehicle group was set as positive control. ***p<0.0001, **p<0.001, *p<0.01 when compared with vehicle group. @@p<0.001, @p<0.05 when compared with Remembrance 500 mg group.

FIG. 56 shows mean escape Latency (n=10) except n=9 in sham group.

FIG. 57 is a graph of rate of loss in time over 5 days.

FIG. 58 is a gel photo of GAPDH gene expression and catalase gene expression in hipocampus (n=3).

FIG. 59 is a graph GST activity in the hippocampus and cortex of the brain (n=3).

FIG. 60 is a graph of SOD activity in the hippocampus and cortex of the brain (n=3).

FIG. 61 is a graph of DNA damage in blood from different groups (n=7).

FIG. 62 is a graph of DNA damage in brain tissue from different groups (n=7).

FIG. 63 is a graph showing mortality of rats myocardial infarcted rats treated with vehicle or various doses of Remembrance.

FIG. 64 is a graph showing infarct size in myocardial infarcted rats treated with vehicle or various doses of Remembrance.

### DETAILED DESCRIPTION OF THE INVENTION

The brain is the major part of central nervous system; it controls many important activities like thoughts, memory, cognitive and other vital physiological activities such as heart rate and respiration. Since all activities are performed by brain cells, their health is very important in maintaining our body's functions. However, brain cells are sensitive cells and susceptible to damage when there is poor circulation supplying oxygen and nutrients.

Leveraging the knowledge and deep understanding of Traditional Chinese medicine (TCM) and physiology of the brain and many years of practice, the inventor discovers a unique methodology for maintaining and promoting brain health, and preventing and treating brain disorders by using innovative combinations of herbal extracts.

The present invention provides novel compositions for pharmaceutical or nutraceutical use in an animal, preferably in a human.

In one aspect, compositions are provided, preferably for promoting brain health, preventing or treating brain disorders. The composition comprises at least 2 herbal ingredients selected from the group consisting of Rhodiola rosea, Ginkgo biloba, Panax notoginseng, and Ligusticum chuanxiong. Preferably, the composition comprises at least 3 herbal ingredients selected from the group consisting of Rhodiola rosea, Ginkgo biloba, Panax notoginseng, and Ligusticum chuanxiong. More preferably, the composition comprises Rhodiola rosea (root), Ginkgo biloba (leaf), Panax notoginseng (root), and Ligusticum chuanxiong (rhizome).

The inventor believes that these four herbal components each has a different mechanism for its anti-oxidant properties, thus scavenging free radicals and hence postulated to prevent apoptosis. As demonstrated in the Example section, the combination of these herbs allows the enhancement of their original functions, which may promote synergism among the component herbs, as well as to decrease any toxic effects of the constituent herbs.

Folium Ginkgo consists of the dried whole leaf of Ginkgo biloba. The free radical scavenging effect of Ginkgo biloba has been demonstrated by the reductions in stroke infarct volume in mice after MCAO as well as the delayed neuronal death in the CA1 region of the hippocampus using a very high dose of Ginkgo biloba in Mongolian gerbils. The exact neuroprotective mechanism of Ginkgo biloba is not known, but it is proposed that Ginkgo biloba composes of flavone glycosides (which is made up of quercetin, kaempferol, rutin and myricetin) as well as terpene lactones (ginkgolides A and B), all which decrease free radical release. Terpene lactones has been shown to improve blood flow and reduce thrombus formation by inhibiting platelet-activating factor.

Radix Rhodiolae belongs to the genus Rhodiolae, a Chinese herb which has its origins from alpine plants, and include a range of antioxidant compounds such as p-tyrosol, organic acids (chlorogenic acid, caffeic acid and gallic acid) as well as flavonoids (catechins and proanthocyanidins).

Rhodiola rosea is also called Russian Rhodiola, which is a powerful anti-aging phyto supplement with adaptogenic and anti-stress activity. In Russia, Rhodiola rosea also known as “Golden root.”

Liguistici chuanxiong (Rhizoma) belongs to the family of Umbelliferae that can improve the microcirculation of the brain by inhibiting thrombus formation, platelet aggregation and blood viscosity. One of the major ingredients in Rhizoma liguistici chuanxiong is ferulic acid, a flavonoid component with antioxidant properties.

Radix notoginseng, also known by its Latin name as Panax notoginseng, contains panaxatriol saponins and has been shown to be neuroprotective by alleviating cerebral edema, up-regulating the expression of heat shock protein 70, down-regulating transferrin and maintaining blood-brain barrier.

Rhodiola rosea, Ginkgo biloba, Panax notoginseng, and Ligusticum chuanxiong may be extracted with alcohol, water, or alcohol/water and the extracts can be concentrated, and dried to solid, such as in a form of powder. Each may be undergo one, or alternatively, two extraction process. Preferably, Rhodiola rosea (root) is extracted with alcohol (ethanol), concentrated, and dried to yield yellowish brown powder with thin odor and bitter taste. In a preferable embodiment of the invention, Rhodiola rosea (root) may go through an extraction process twice, each time with alcohol and water. Ginkgo biloba (leaf) is extracted with water/alcohol, concentrated, and dried to yield light brownish yellow powder with thin odor and bitter taste. Panax notoginseng (root) is alcohol extracted, concentrated, and dried to yield yellowish brown powder with thin odor and bitter and sweet taste. In a preferable embodiment of the invention, Panax notoginseng (root) is extracted with alcohol and water. Ligusticum chuanxiong (rhizome) is extracted with water/alcohol, concentrated, and dried to yield yellowish brown powder with thick odor and mild bitter taste.

The concentration of Rhodiola rosea is about 5-95%, 10-90%, 30-90%, 40-85%, 50-85%, 60-80%, or 65-75% w/w based on the total weight of the composition.

The concentration of Ginkgo biloba is about 5-50%, 5-40%, 7-35%, 10-30%, or 10-20% w/w based on the total weight of the composition.

The concentration of Panax notoginseng is about 5-50%, 5-40%, 7-35%, 10-30%, or 10-20% w/w based on the total weight of the composition.

The concentration of Ligusticum chuanxiong is about 1-50%, 1-40%, 2-35%, 3-30%, 3-8%, 3-6%, or 3-5% w/w based on the total weight of the composition.

In a preferred embodiment, the composition is a combination of extracts of Rhodiola rosea at about 50-85%; Ginkgo biloba at about 10-30%; Panax notoginseng at about 10-20%; and Ligusticum chuanxiong at about 3-8% w/w based on the total weight of the composition.

In a particular embodiment, the composition is a combination of extracts of Rhodiola rosea (root) at about 75%; Ginkgo biloba (leave) at about 10%; Panax notoginseng at about 10%; and Ligusticum chuanxiong (rhizoma) at about 5% w/w based on the total weight of the composition. This composition is designated as Formulation A in the Example section below.

In a particular embodiment, the composition is a combination of extracts of Rhodiola rosea (root) at about 50%; Ginkgo biloba (leave) at about 35%; Panax notoginseng at about 10%; and Ligusticum chuanxiong (rhizoma) at about 5% w/w based on the total weight of the composition. This composition is designated as Formulation B in the Example section below.

The composition may further comprise Danshen (Salvia militorrhiza) at about 1-50%, 1-40%, 2-35%, 3-30%, 3-8%, 3-6%, or 3-5% w/w based on the total weight of the composition. Optionally, Salvia militorrhiza may substitute Ligusticum chuanxiong in the composition.

The composition preferably contains minimum amount of water, more preferably containing less than 0.5% of water by weight, and most preferably containing less than 0.1% water by weight.

For oral administration, the inventive composition can be formulated readily by mixing the herbal ingredient, optionally in combination with physiologically or pharmaceutically acceptable carriers that are well known in the art. Such carriers enable the herbal ingredients to be formulated as tablets, pills, dragees, capsules, emulsions, lipophilic and hydrophilic suspensions, liquids, gels, syrups, slurries, suspensions and the like, for oral ingestion by an individual or a patient to be treated.

In a preferred embodiment, the inventive composition is contained in capsules. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. All Formulations for oral administration should be in dosages suitable for such administration. Preferably, each capsule contains about 100-1000 mg, 100-800 mg, 200-600 mg, 300-500 mg of a mixture of extracts of Rhodiola rosea at about 50-85%; Ginkgo biloba at about 10-30%; Panax notoginseng at about 10-20%; and Ligusticum chuanxiong at about 3-8% w/w based on the total weight of the composition. It shall be understood that these and other alternate embodiments of the invention may include capsules formed of materials besides gelatin such as vegetarian based capsules made from hydroxypopylmethylcellulose.

Optionally, the inventive composition for oral use can be obtained by mixing the inventive composition with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal administration, the inventive compositions may take the form of tablets or lozenges formulated in conventional manner.

For administration by inhalation, the inventive composition for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In another aspect of the invention, a method of promoting blood circulation in the brain is provided. The method comprises: administering to a mammal in need of promotion of blood circulation in the brain any of the inventive compositions described above. The mammal is preferably a human.

In yet another aspect of the invention, a method of promoting brain health is provided. The method comprises: administering to a mammal in need of promotion of brain health any of the inventive compositions described above. The mammal is preferably a human.

In yet another aspect of the invention, a method of restoring or improving memory is provided. The method comprises: administering to a mammal in need of restoration or improvement of memory any of the inventive compositions described above. The mammal is preferably a human.

In still another aspect of the invention, a method of preventing or reducing the risk of developing brain disorder is provided. The method comprises: administering to a mammal in need of such prevention or at risk of developing brain disorder any of the inventive compositions described above. The mammal is preferably a human. The brain disorder is stroke such as ischemic stroke or a neurodegenerative disease such as Huntington's disease, Alzheimers's disease, and amyotrophic lateral sclerosis.

The present invention also provides a kit or assembly of kits containing the inventive composition. The kit may contain the composition, preferably a combination of Rhodiola rosea, Ginkgo biloba, Panax notoginseng, and Ligusticum chuanxiong in a uniform dosage form in a vessel. The kit may further comprise instruction as to how to use the kit for promoting brain health, treating or preventing a disease or condition described above, such as memory loss, ischemic stroke, and a neurodegenerative disease. The instruction may be in a printed form.

The amount of the inventive composition administered will, of course, be dependent on the subject being treated, the subject's weight, the manner of administration and the judgment of the prescribing physician. Generally, however, the dosage of the inventive composition will be about 0.01 mg/kg/day to about 1000 mg/kg/day, about 0.01 mg/kg/day to about 500 mg/kg/day, about 1 mg/kg/day to about 600 mg/kg/day, about 10 mg/kg/day to about 500 mg/kg/day, about 20 mg/kg/day to about 300 mg/kg/day, or about 50 mg/kg/day to about 200 mg/kg/day. Preferably, the dosage of the inventive composition is about 2-100 mg/kg/day, 5-50 mg/kg/day, 7-40 mg/kg/day, or 8-25 mg/kg/day.

For example, for the prevention of brain ischemia, the preferred dosage of the inventive composition is about 10-100 mg/kg, 20-60 mg/kg, or 30-50 mg/kg once or twice a day.

For example, for the prevention of myocardial infarction (MI), the preferred dosage of the inventive composition is about 10-200 mg/kg, 20-100 mg/kg, or 40-80 mg/kg once or twice a day.

The inventive composition may also be combined with another therapeutic agent (e.g., Losartan, Simvastin, Ramipril, Aspirin, TPA and the like) or nutritional supplement (e.g., ginkgo, Lingzhi, green tea, vitamins) to prevent or treat diseases and conditions described above additively or synergistically, such as ischemic stroke, neurodegenerative diseases such as Huntington's disease and Alzheimers's disease, and cardiovascular diseases such as amyotrophic lateral sclerosis.

### EXAMPLES

### Example 1

### Effect of Formulation A on Ischemic Stroke Rat Model: a Chronic Study

This study evaluated and investigated the effect of chronic treatment of Formulation A of the present invention compared to well-known western drugs and other substances (See Table 1.) in an ischemic stroke model using Wistar rats with middle cerebral artery occlusion (MCAO).

### Methodology:

Formulation A and other comparative drugs were administrated orally once daily for two months to Wistar rats Table 1 lists the dosage of administration. Stroke was induced by occlusion of middle cerebral artery (MCA). Formulation A and the drugs were continued for another month. At the end of treatment period, all rats were scarified by decapitation after measuring the hemodynamic parameters. Brains and livers were collected for further studies.

TABLE 1
Dosage of administration

| Treatment | Dosage | | Number of Rats |
|---|---|---|---|
| Sham | — | | 12 |
| Sham + Formulation A | 500 | mg/kg/day | 12 |
| Vehicle | 4 | ml/kg/day | 12 |
| Formulation A | 250 | mg/kg/day | 12 |
| Formulation A | 500 | mg/kg/day | 12 |
| Ginkgo | 20 | mg/kg/day | 12 |
| Aspirin | 10 | mg/kg/day | 12 |
| Losartan | 1 | mg/kg/day | 12 |

N = 96 Wistar Rats

### Mortality Rate:

The mortality rates of the rats with ischemic stroke in different treatment groups were compared.

### Clinical Evaluation of Ischemia by Neurological Scores:

A neurological scale of 0 to 5 was used to assess the motor and behavioral changes observed in the ischemic rats after the MACO procedure. The evaluation scale is summarized in Table 2.

TABLE 2
Neurological evaluation of rats after MACO

| Score | Evaluation |
|---|---|
| 0 | No apparent deficit |
| 1 | Failure to extend contralateral forepaw fully |
| 2 | Spontaneous movement in all directions; contralateral |
| | circling only if pulled by tail |
| 3 | Spontaneous contralateral circling |
| 4 | Inactive, walk only when stimulated |
| 5 | Dead |

### Infarct Volume Assessment:

The area of cerebral infarction was quantified with TTC (2,3,5-triphenyltetrazolium chloride) staining. The cerebrum was removed and cleared of all overlying membranes. It was sectioned into 8 pieces of 2-mm thick coronal slices, and then stained with 0.1% TTC solution at 37 deg. C. for 30 minutes. Healthy brain tissue would turn purple on staining while the infarct area due to ischemia left a white unstained area. The infarct size would be analyzed by an image analyzer system (Scion image for windows, Beta 4.0.2) and converted by integration to the true infarct size of ischemia damage.

### Results:

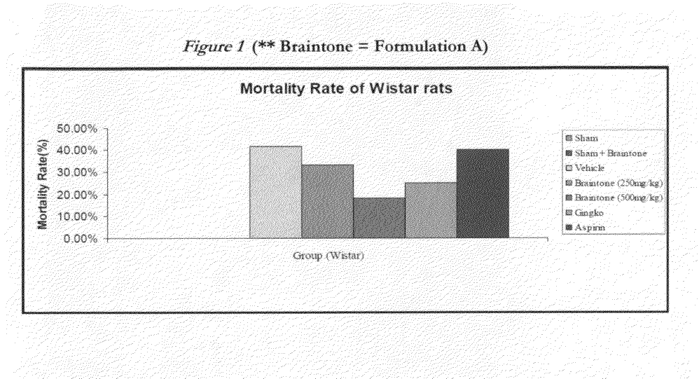

1. Formulation A-treated group (500 mg/kg/day) had the lowest mortality rate (20%) after MACO compared to other treatment groups. (FIG. 1: mortality rate of Wistar rats)

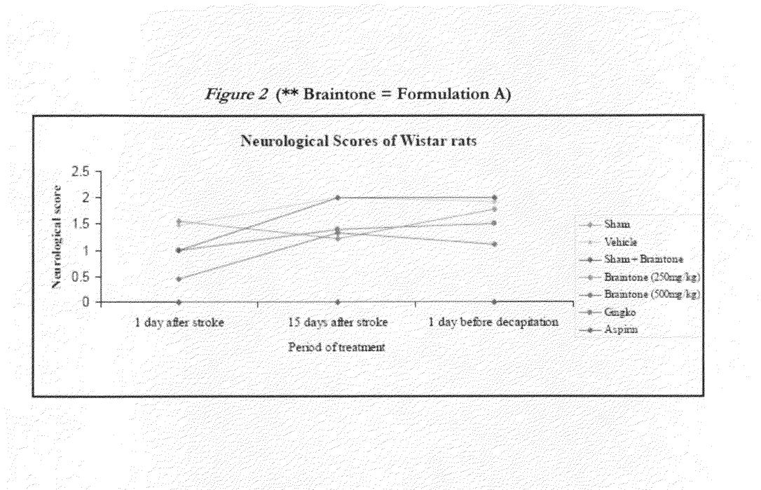

2. Formulation A-treated groups (250 and 500 mg/kg/day) also had the lowest neurological score compared to other treatment groups on the 15th day after MCAO. (Low neurological score means less damage of neuron cells). (FIG. 2: Neurological scores of Wistar Rats)

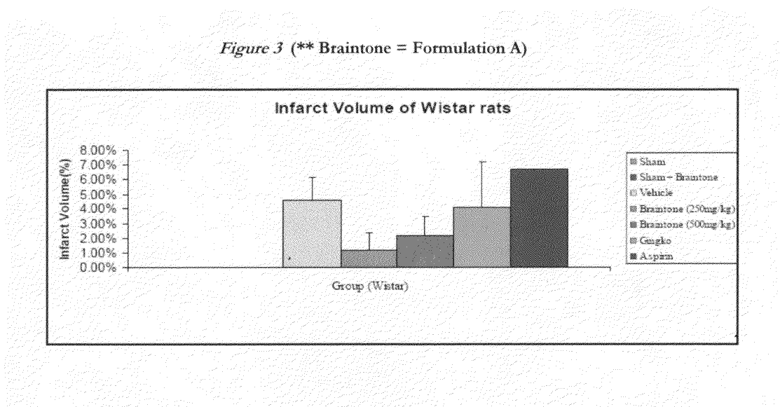

3. Formulation A-treated groups (250 and 500 mg/kg/day) had the lowest infarct volume compared to the other treatment groups. (FIG. 3: Infarct volume of Wistar rats)

### Conclusions:

The brain is the major part of central nervous system; it controls many important activities like thoughts, memory, cognitive and other vital physiological activities such as heart rate and respiration. Since all activities are performed by brain cells, their health is very important in maintaining our body's functions. However, brain cells are sensitive cells and susceptible to damage when there is poor circulation supplying oxygen and nutrients.

The experimental results show the infarct volume after MCAO in Wistar rats' brains appears significantly lower in Formulation A-treated groups (250 and 500 mg/kg/day), which indicates that Formulation A may reduce the area of tissue necrosis associated with ischemia. Formulation A-treated groups (250 and 500 mg/kg/day) had lower neurological scores than other treatment groups indicating better neurological function after an ischemic event. Additionally, Formulation A (500 mg/kg/day) had the lowest mortality rate when compared to ginkgo, aspirin or the control vehicle group after MCAO. These results suggest that Formulation A may better preserve neurological function and blood circulation in the face of an ischemic event compared to ginkgo or aspirin alone. Good circulation of blood and nutrients to the brain ensures the continued health of neuron cells, and therefore helps maintain brain cognitive and memory functions.

By maintaining and preserving adequate blood circulation to the brain, Formulation A can help ensure a sufficient supply of oxygen and nutrients to neuron cells enabling healthy memory and cognitive functions as well as mitigating the area of damage related to an ischemic event in the brain if used preventatively.

### Example 2

### Effect of Formulation A of the Present Invention on Memory: a Chronic Study

### Objectives:

The study aims to investigate the treatment effect of Formulation A in a Sprague Dawley rat memory impairment model compared with sham, vehicle, simvastatin, lingzhi (ganoderma lucidum) and ginkgo control groups.

### Methodology:

The Morris Water Maze Task, a well-known animal model developed to study the learning ability in animals, was used for testing the memory function of the Sprague Dawley rats weighing 250-300 g. This task uses a round water pool divided into four zones with a platform submerged beneath the surface in zone four. When placed in the maze, the rat's task is to find the hidden platform in the spatial acquisition test after being given scopolamine to induce memory impairment. Escape latency, distance swam and speed of swim are recorded during the spatial acquisition test.

A second probe trial test on day 6 was used to measure how much time each rat swam in each zone of the water pool once the submerged platform is removed. Two hours after the probe trial is completed the rates are decapitated and their brains are collected for further study measuring DNA damage, gene expression, and antioxidant assays. Molecular analysis also includes immunohistology testing and tunnel staining methods.

### Treatment Timeline:

Each rat treatment group consists of 10 rats (See table 3 for treatment groups and drug dosages.) Each treatment group is pretreated with their designated drugs from day 1-14. On days 1 and 7 pretraining of the rats in the water maze occurs. During pretraining days 1 and 7, each rat has two training sessions a day with 30 minutes rest in between each swim whereby they are given 120 sec. to find the submerged platform in zone four when placed in the maze randomly. If they are unable to find the platform after 120 sec then they are guided to the platform.

On days 15-19 the spatial acquisition test is carried out at the same time each day. Sixty minutes prior to the test, each rat is given their assigned drug according to their treatment group and 30 minutes prior they are given 1 mg/kg of scopolamine to induce memory impairment. (See Table 4.) During each daily spatial acquisition test, each rat is given four consecutive trials of 120 sec. with 30 minutes rest in between to find the submerged platform when starting from a random point within the maze. Escape latency, distance swam and speed of swim are recorded during each trial.

On day 20, after rats are given their assigned drug according to their treatment group the platform is removed from the maze and the probe trial is done whereby rats are given 120 sec to swim in the maze. Time spent and distance swam in each zone are recorded.

Two hours after the probe trial is complete, the rats are decapitated and brains are collected for further study measuring DNA damage, gene expression, and antioxidant assays. Molecular analysis also includes immunohistology testing and tunnel staining methods.

Results: (n=5 in each treatment group)

### Spatial Acquisition Test:

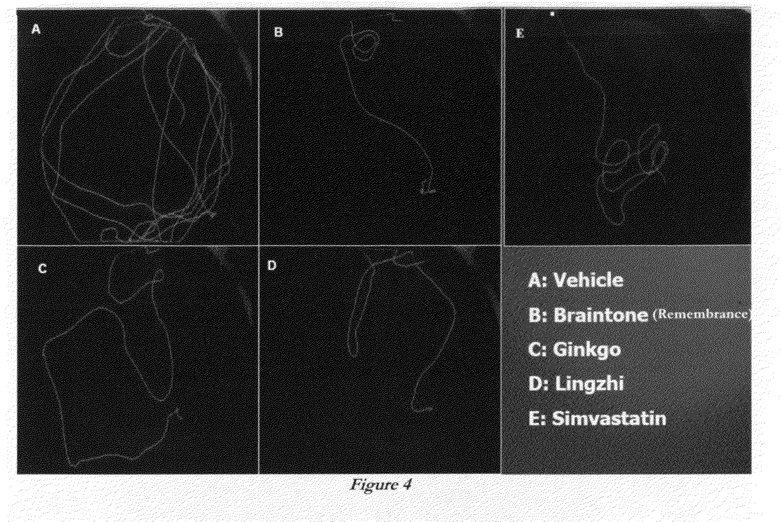

1. Swim Patterns (FIG. 4):

Formulation A-treated rats (500 mg/kg/day) showed a simple swim pattern in the Morris Water Maze Task when compared to the vehicle group. For this parameter only one rat in each group is shown.

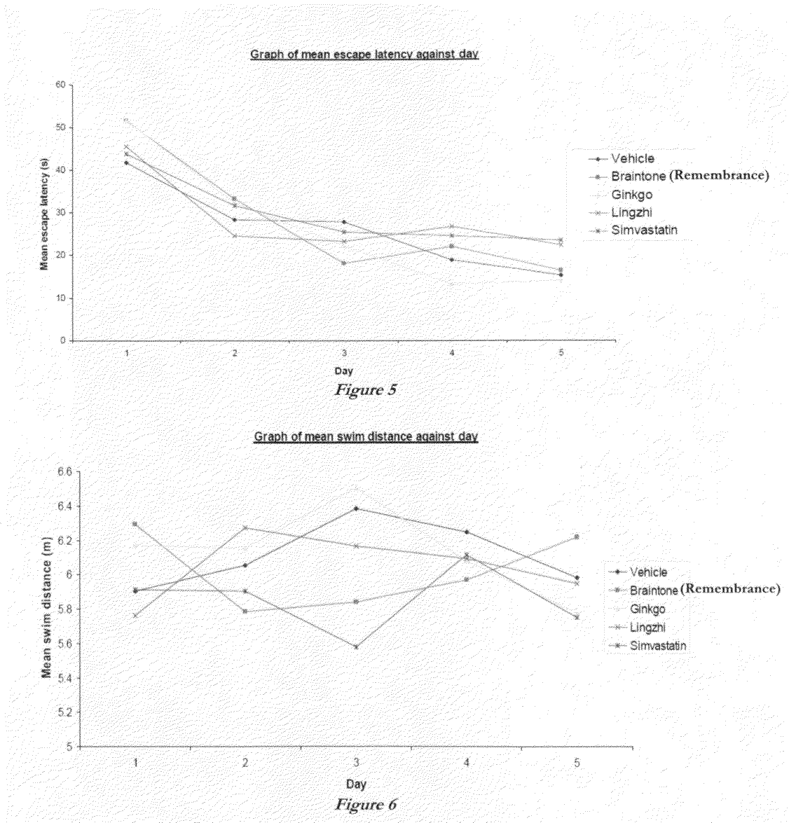

2. Mean Escape Latency (FIG. 5):

Formulation A-treated group (500 mg/kg/day) had the lowest mean escape latency compared to all other treatment groups on day three and was lower than lingzhi and simvastatin on day 5.

3. Mean Swim Distance (FIG. 6):

Formulation A-treated group (500 mg/kg/day) had the lowest mean swim distance on day 2 compared to all other groups and was lower than the gingko, lingzhi and vehicle groups on days 3. This effect was not noted on day 5.

4. Mean Swim Speed (FIG. 7):

Formulation A-treated group (500 mg/kg/day) generally swam faster compared to other treatment groups.

### Probe Test:

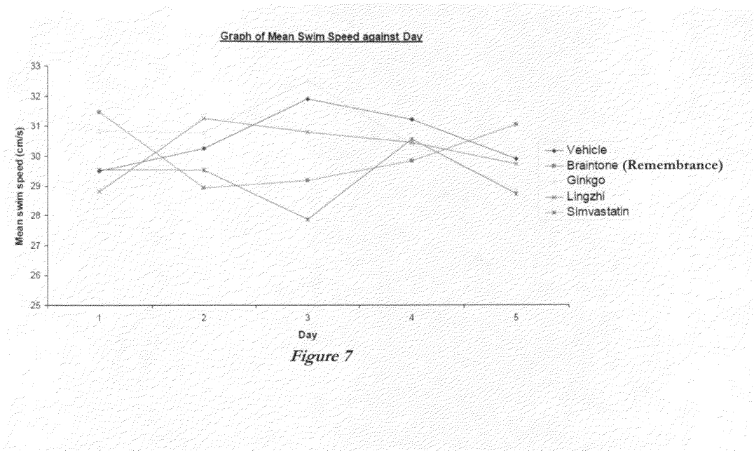

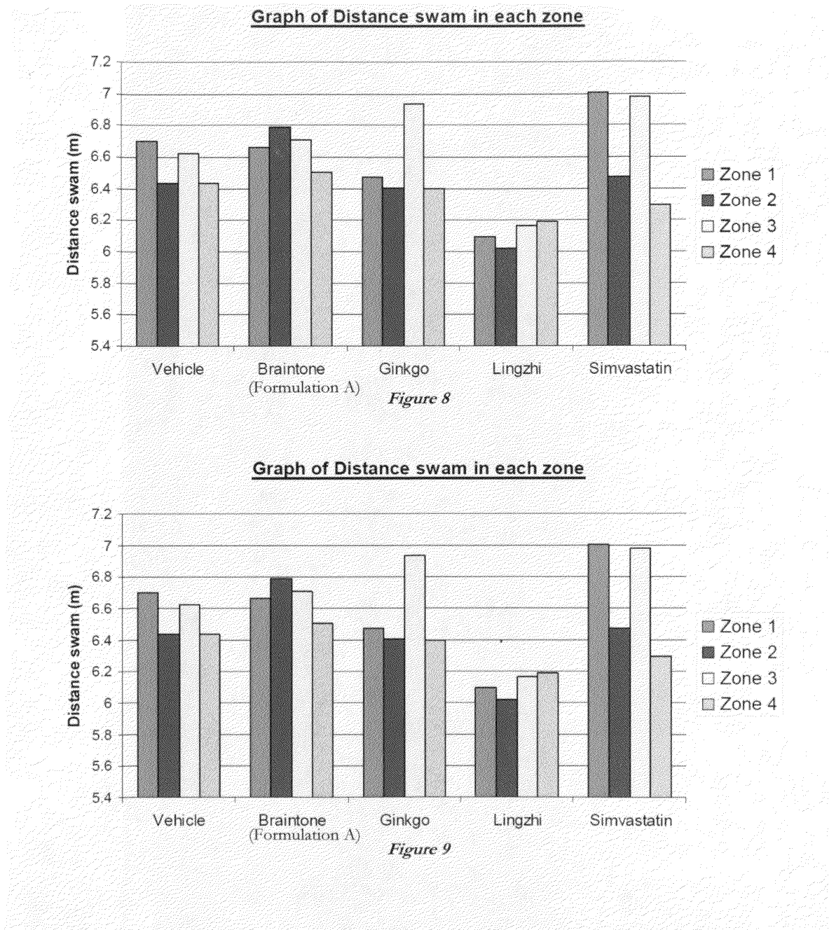

1. Distance Swam in Each Zone (FIG. 8):

The Formulation A treated group swam more distance in zone four compared to ginkgo and lingzhi and slightly more than the vehicle group.

2. Time Spent in Different Zone (FIG. 9):

Formulation A-treated group (500 mg/kg/day) spent a longer time in zone 4 compared to gingko and lingzhi but in general all groups spent a similar time in zone 4. (Zone 4 is the region where the platform was originally located.)

### Conclusions:

These results show that rats treated with Formulation A (500 mg/kg/day) for 20 days had similar performance on the water maze task compared to other groups in the special acquisition test and slightly better performance on the probe test with regards to speed and time in zone four compared with other treatment groups suggesting possible enhanced learning ability resulting form treatment with Formulation A. This supports the results found in EXAMPLE 1 that Formulation A may better preserve neurological function and blood circulation compared to ginkgo alone. One theory for better performance on the water maze task compared to other groups may be attributed to Formulation A's ability to promote blood circulation and delivery of nutrients to the brain in order to ensure the continued health of neuron cells, thereby maintaining brain cognitive and memory functions.

TABLE 3
Oral treatment of Wistar rats

| | | | Number |
|---|---|---|---|
| Group | Treatment | Dosage | of rats |
| Sham | | | 10 |
| Vehicle | Distilled water | | 10 |
| Braintone/ | Braintone/ | 500 mg/kg/day | 10 |
| Formulation A | Formulation A | | |
| Ginkgo | Ginkgo biloba extract | 100 mg/kg/day | 10 |
| Lingzhi | Winsor lingzhi extract | 500 mg/kg/day | 10 |
| Simvastatin | Simvastatin | 10 mg/kg/day | 10 |

TABLE 4
Oral treatment of Sprague Dawley rats
Sprague Dawley rats, 250-300 g

| | | | Number |
|---|---|---|---|
| Group | Treatment | Dosage | of rats |
| Sham | | | 10 |
| Vehicle | Distilled water | | 10 |
| Braintone | Braintone | 500 mg/kg/day | 10 |
| (Remembrance) | (Remembrance) | | |
| Ginkgo | Ginkgo biloba extract | 100 mg/kg/day | 10 |
| Lingzhi | Winsor lingzhi extract | 500 mg/kg/day | 10 |
| Simvastatin | Simvastatin | 10 mg/kg/day | 10 |

### Example 3

### Long-Term Toxicological Study for Formulation A of the Present Invention in Wistar Rat Model

### Objectives:

This study evaluated the toxicological effect of long-term administration of Formulation A in small animals (Wistar rat model).

### Methodology:

Performed simultaneously with example one, Formulation A and other comparative drugs were administrated orally once daily for two months to Wistar rats (see table 5). After two months rats underwent MCAO except the sham treatment groups and continued on their treatment regimens for an additional month for a total of three months. All observations were recorded after the 3-month treatment period. The general condition of the animals such as body weight, breathing, CNS reactions, movement of legs and dysfunction in the mucosa of the rats' eyes and mouths were monitored. Blood samples were taken to determine the full blood count (see table 6), liver function, renal function and pancreatic function. Wistar rats were sacrificed at the end of the three month treatment period and post-mortem pathological studies were performed on the heart, lungs, liver, spleen and kidneys by using light and electron microscopy analysis.

TABLE 5
Dosage of administration

| Treatment | Dosage | Number of Rats |
|---|---|---|
| Sham | — | 12 |
| Sham + Formulation A | 500 mg/kg/day | 12 |
| Vehicle | 20 mg/kg/day | 12 |
| Formulation A/Braintone | 250 mg/kg/day | 12 |
| Formulation A/Braintone | 500 mg/kg/day | 12 |
| Ginkgo | 20 mg/kg/day | 12 |
| Aspirin | 10 mg/kg/day | 12 |
| Losartan | 1 mg/kg/day | 12 |
| White Tea | 100 mg/kg/day | 12 |

TABLE 6
Full blood count

| Parameters | Descriptions |
|---|---|
| White blood cell | |
| Red blood cell | |
| Hemoglobin | |
| Mean corpuscular volume (MCV) | Average red blood cell volume |
| Mean corpuscular hemoglobin (MCH) | Measure of the amount of |
| | hemoglobin in RBC |
| Mean corpuscular hemoglobin conc. | Measure of the concentration |
| (MCHC) | of hemoglobin in RBC |
| Hematocrit | Packed red blood cell volume |
| Mean platelet volume (MPV) | Average size of platelet in blood |
| Platelet | |
| Red blood cell distribution width | Variation of RBC volume |
| (RDW) | |

### Full Blood Count:

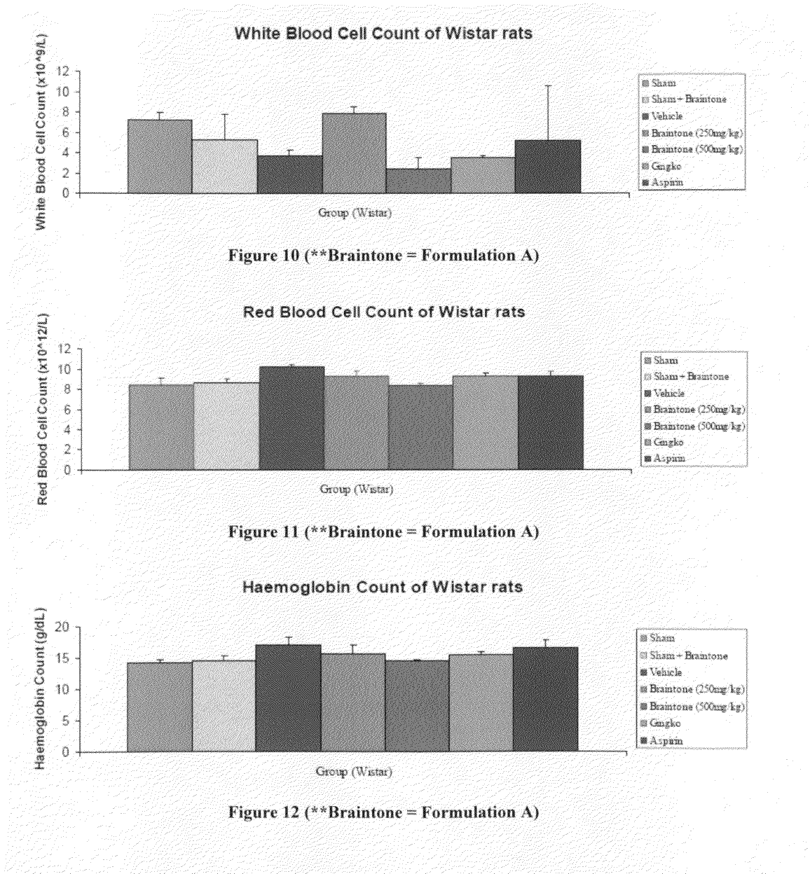

White blood cell count: Formulation A-treated group (250 mg/kg/day) did not affect the white blood cell count compared to the sham group. (FIG. 10)

Red blood cell count: Formulation A-treated groups (250 and 500 mg/kg/day) did not affect the red blood cell count compared to other treatment groups. (FIG. 11)

Hemoglobin count: Formulation A-treated groups (250 and 500 mg/kg/day) did not affect the hemoglobin count compared to other treatment groups. (FIG. 12)

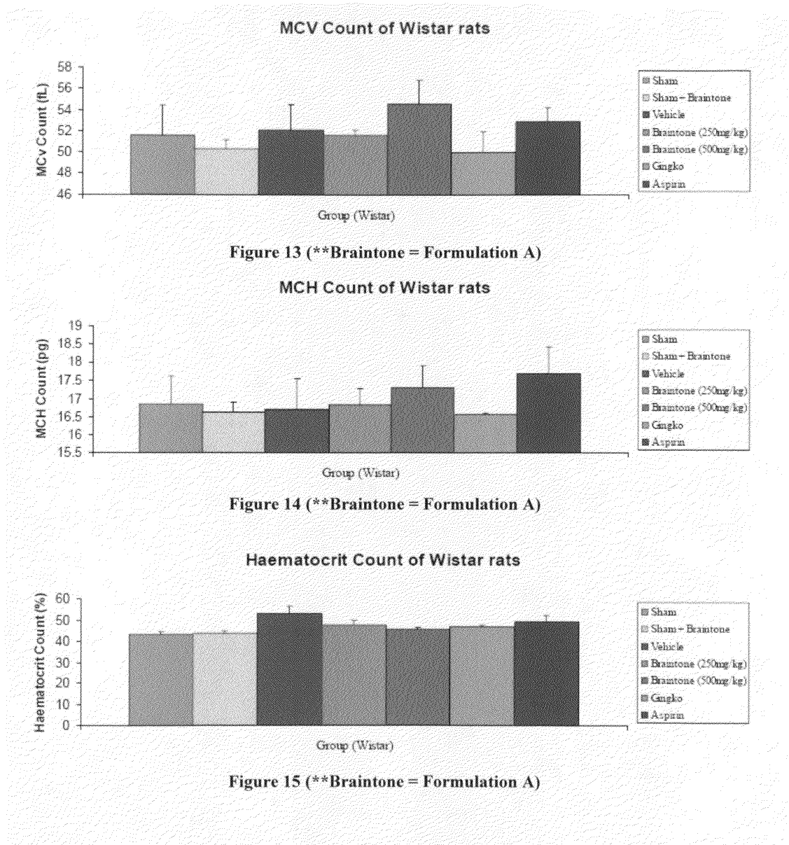

MCV: Formulation A-treated group (250 mg/kg/day) did not affect MCV count in blood compared to the sham and vehicle groups. (FIG. 13)

MCH: Formulation A-treated group (250 mg/kg/day) did not affect MCH count in blood compared to the sham and vehicle groups. (FIG. 14)

Hematocrit: Formulation A-treated groups (250 and 500 mg/kg/day) did not affect the hematocrit count compared to other treatment groups. (FIG. 15)

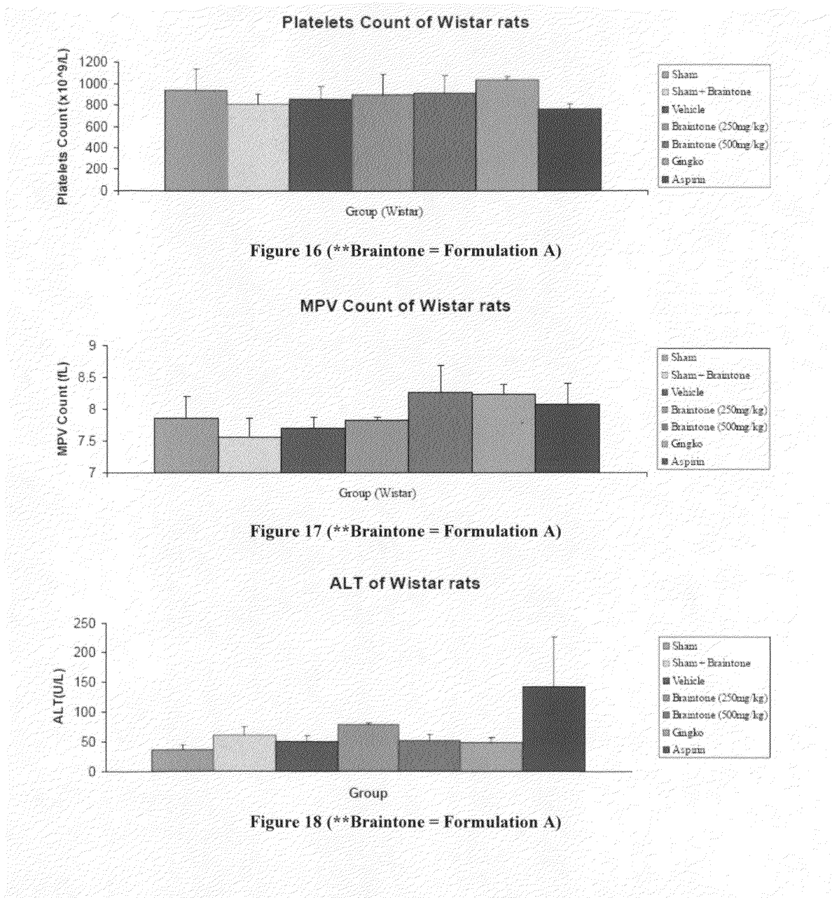

Platelet: Formulation A-treated groups (250 and 500 mg/kg/day) did not affect the platelet count compared to other treatment groups. (FIG. 16)

MPV: Formulation A-treated group (250 mg/kg/day) did not affect MPV in blood compared to the sham and vehicle groups. (FIG. 17)

### Liver Functions:

ALT: Both Formulation A-treated groups (250 and 500 mg/kg/day) did not affect the level of GPT in blood compared to other treatment groups. (FIG. 18)

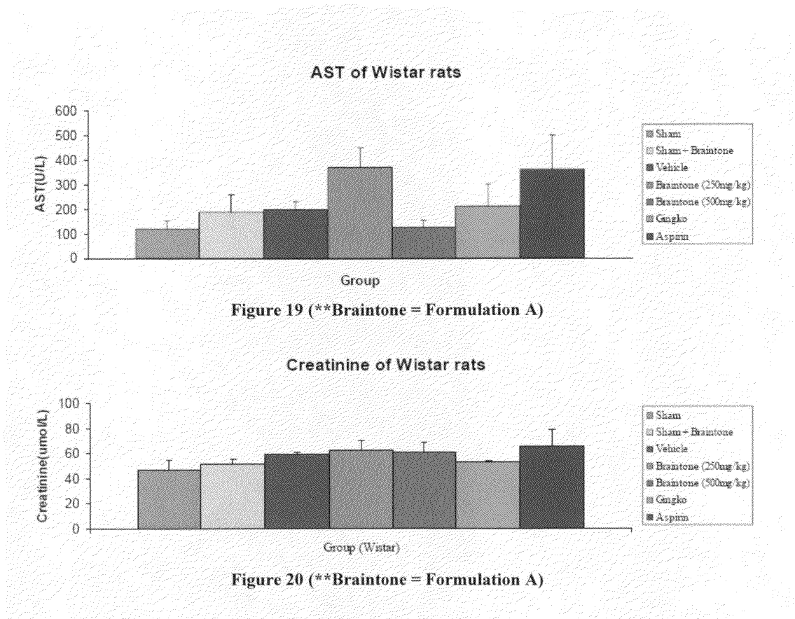

AST: Formulation A-treated groups (500 mg/kg/day) did not affect the level of GOT in blood compared to the Sham group. (FIG. 19)

### Renal Functions:

Creatinine: Both Formulation A-treated groups (250 and 500 mg/kg/day) did not affect the level of creatinine in blood compared to other treatment groups. (FIG. 20)

### Pancreatic Functions:

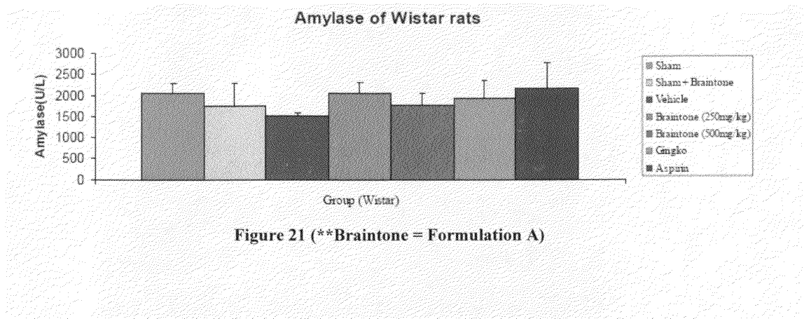

Amylase: Both Formulation A-treated groups (250 and 500 mg/kg/day) did not affect the level of amylase in blood compared to other treatment groups. (FIG. 21)

### Conclusion:

Overall it appears the full blood count, liver, renal and pancreatic functions of the Wistar rats are not affected with the long-term treatment of Formulation A at 250 mg/kg/day, ginkgo and other treatment drugs when compared with the sham treatment group.

### Example 4

### Protective Effects of Formulation B of the Present Invention on Stroke Rats: 2-Week Study

### Objective:

To evaluate the effect of Formulation B on Spontaneous Hypertensive Rats (SHR) and Wistar Rats during the acute phase of stroke and in cerebral remodeling phase (MCAO).

### Methodology:

Wistar and SHR rats were randomly divided into four treatment groups and one sham group (see Table 7 and 8).

Formulation B and Losartan were administrated orally once daily for a week before MCAO (middle cerebral artery occlusion) and the administration was continued for another seven days. Eventually, all rats were scarified by decapitation after measuring hemodynamic parameters. In the sham groups, the rats did not have MCAO but underwent a sham operation by which their skull was opened but no MCAO procedure occurred.

### Mortality Rate:

The mortality rates of rats with ischemic stroke in different treatment groups were compared according to the number of rats dead at day 13.

### Clinical Evaluation of Ischemia by Neurological Scores:

A neurological scale of 0 to 5 was used to assess the motor and behavioral changes observed in the ischemic rats after the MACO procedure. The evaluation scale is summarized in Table 3.

TABLE 3
Neurological evaluation of rats after MACO

| Score | Evaluation |
|---|---|
| 0 | No apparent deficit |
| 1 | Failure to extend contralateral forepaw fully |
| 2 | Spontaneous movement in all directions; contralateral |
| | circling only if pulled by tail |
| 3 | Spontaneous contralateral circling |
| 4 | Inactive, walk only when stimulated |
| 5 | Dead |

### Infarct Volume Assessment:

The area of cerebral infarction was quantified with TTC (2,3,5-triphenyltetrazolium chloride) staining. The cerebrum was removed and cleared of all overlying membranes. It was sectioned into 8 pieces of 2-mm thick coronal slices, and then stained with 0.1% TTC solution at 37 deg. C. for 30 minutes. Healthy brain tissue would turn purple on staining while the infarct area due to ischemia left a white unstained area. The infarct size would be analyzed by an image analyzer system (Scion image for windows, Beta 4.0.2) and converted by integration to the true infarct size of ischemia damage.

### Haematoxylin and Eosin Staining (HE Staining):

The morphological changes were evaluated after cerebral ischemia. The brains were dipped in M-1 Embedding Matrix for frozen sectioning, frozen in liquid nitrogen and stored at −80 deg C. The cerebral sections were then cut from the middle part of the frozen brain at 20 um. 4 to 6 sections were obtained from each brain for HE staining. HE staining of sections was performed to differentiate capillaries, after which they were covered with crystal mount. Cerebral sections were examined under the microscope and photographed.

### Results:

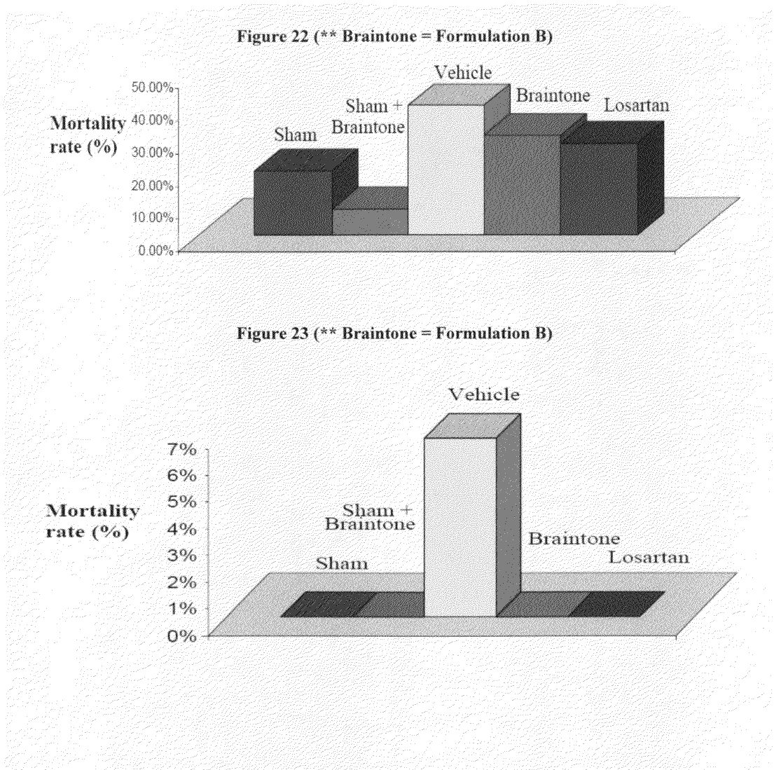

1. Mortality rate of SHR strain (FIG. 22): The ‘SHR Sham’ had mortality of 20% while ‘SHR Sham with treatment of Formulation B (500 mg/kg/day)’ had a mortality rate of 8.3%. The vehicle group had mortality rate of 40% while ‘Formulation B-treated group (500 mg/kg/day)’ had a lower mortality rate of 30.8%. ‘Losartan-treated group (1 mg/kg/day)’ had mortality rate of 28.6%.

2. Mortality rate of Wistar strain (FIG. 23): The ‘Wistar vehicle group’ has mortality rate of 6.7%. While other groups including ‘Formulation B-treated group (500 mg/kg/day)’ and ‘Losartan-treated group (1 mg/kg/day)’ had mortality rate of 0%.

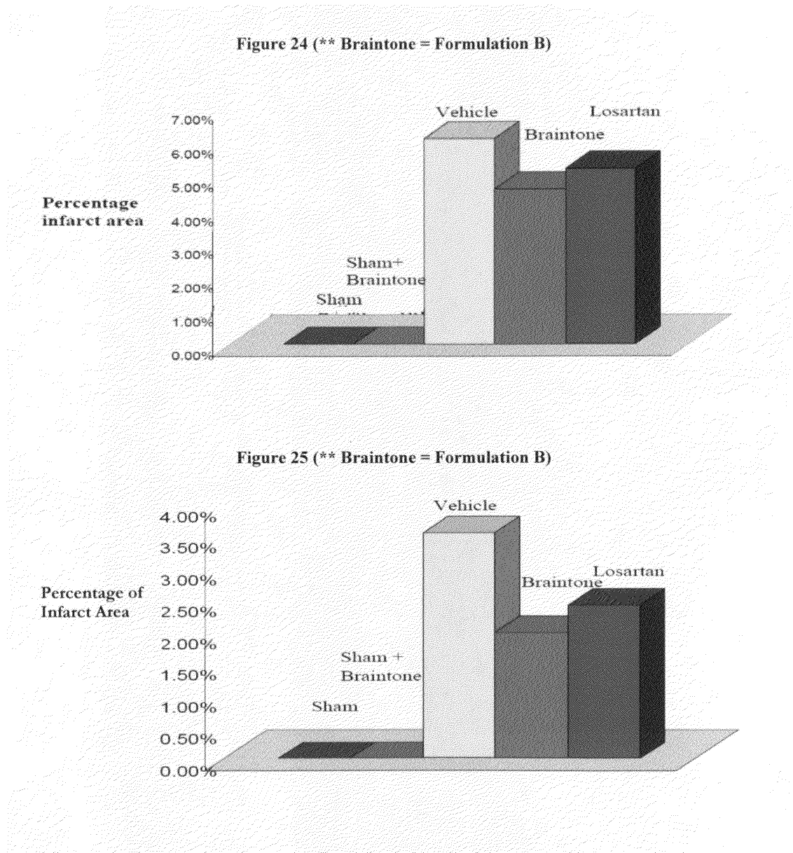

3. Infarct Volume Assessment of SHR strain (FIG. 24): SHR Formulation B-treated group (500 mg/kg/day) had the lowest infarct volume (4.6%) compared to the vehicle (6.1%) and Losartan-treated group (5.22%).

4. Infarct Volume Assessment of Wistar strain (FIG. 25): Wistar Formulation B-treated group (500 mg/kg/day) had the lowest infarct volume (1.98%) compared to the vehicle (3.55%) and Losartan-treated group (2.41%).

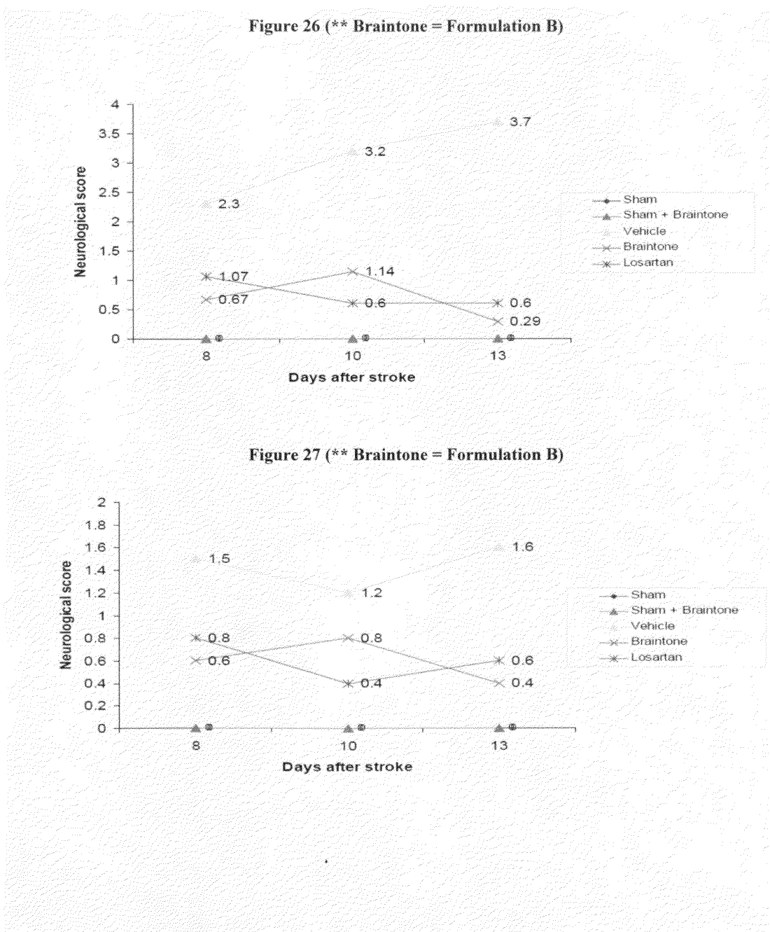

5. Neurological score for SHR strain after stroke (FIG. 26): Formulation B-treated group (500 mg/kg/day) had the lowest neurological score compared to vehicle and Losartan-treated group on day 8 and 13.

6. Neurological score for Wistar strain after stroke (FIG. 27): Formulation B-treated group (500 mg/kg/day) had the lowest neurological score compared to vehicle and Losartan-treated group on day 8 and 13.

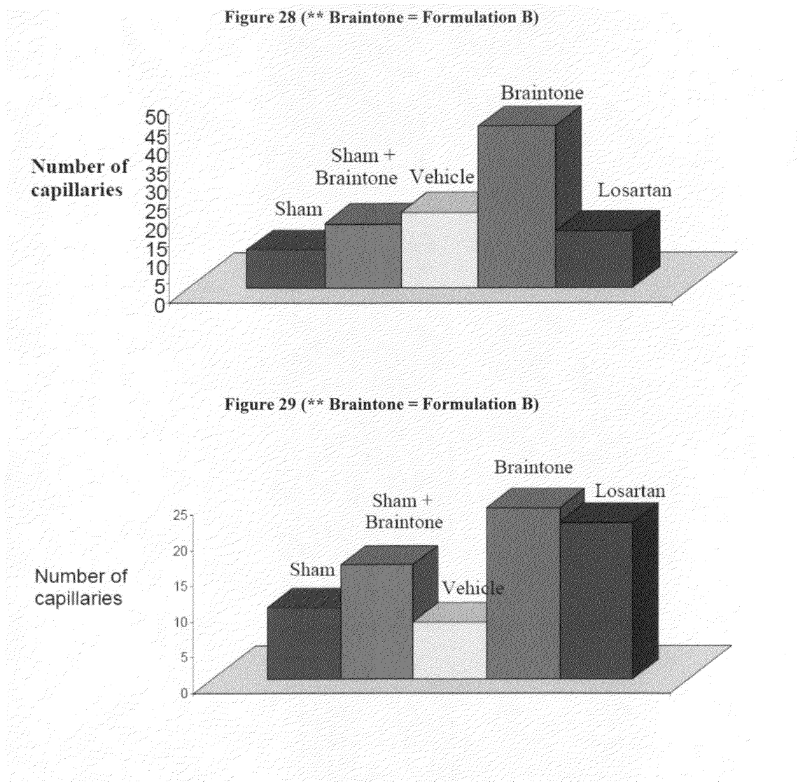

7. HE staining of the number of capillaries for SHR strain (FIG. 28): Formulation B-treated group had the highest number of capillary (43 capillaries) compared to vehicle (20 capillaries) and Losartan-treated group (15 capillaries).

8. HE staining of the number of capillaries for Wistar strain (FIG. 29): Formulation B-treated group had the highest number of capillary (24 capillaries) compared to vehicle (8 capillaries) and Losartan-treated group (22 capillaries).

### Conclusions:

The experimental results show the infarct volume after MCAO in Wistar rats' and SHR's brains appears lower in Formulation B-treated groups (500 mg/kg/day), which indicated that Formulation B may reduce the area of tissue necrosis associated with ischemia. Formulation B-treated groups had lower neurological scores than other treatment groups indicating better neurological function after an ischemic event (day 8 and day 13). Additionally, Formulation B (500 mg/kg/day) had the lowest mortality rate when compared with other treatment groups in both MCAO Wistar rats and SHR. Formulation B-treated groups also had the highest number of capillaries among the other groups.

These results suggest that Formulation B may better preserve neurological function and blood circulation in the face of an ischemic event and hypertension compared to the western medicine, losartan. Good circulation of blood and nutrients to the brain ensures the continued health of neuron cells, and therefore helps maintain brain cognitive and memory functions.

TABLE 7
SHR Rats Group

| Groups | Dosage | Number of Rats |
|---|---|---|
| Sham | — | 15 |
| Sham + Formulation B | 500 mg/kg/day | 15 |
| Vehicle | 20 mg/kg/day | 15 |
| Formulation B | 500 mg/kg/day | 15 |
| Losartan | 1 mg/kg/day | 15 |

TABLE 8
Wistar Rats Group

| Groups | Dosage | Number of Rats |
|---|---|---|
| Sham | — | 15 |
| Sham + Formulation B | 500 mg/kg/day | 15 |
| Vehicle | 20 mg/kg/day | 15 |
| Formulation B | 500 mg/kg/day | 15 |
| Losartan | 1 mg/kg/day | 15 |

### Example 5

### Comparison of Formulation B with Ginkgo, Losartan and Ramipril: an Antioxidant and DNA Damage Study for Cerebral Protection

### Objective:

Antioxidant capacities of Formulation B and its effect on the amount of DNA damage were compared with select single herbal ingredients and Ramipril and Losartan. The role of Formulation B in cerebral protection was discussed.

### Methodology:

### Inhibition of Pyrogallol Red Bleaching by Hypochlorous Acid (HOCL)

Pyrogallol Red was dissolved in 100 mM buffer at 37 deg. C., pH 7.4, after which 125 uM HOCL was added and incubated with 10 mg/ml ascorbic acid and other select herbal extracts including two batches of Formulation B. The absorbance values of the mixtures in the presence and absence of the antioxidant were recorded by UV visible spectrophotometer at 542 nm.

### Inhibition of ABTS Assay

10 ul of 10 mg/ml of select herbal extracts and two batches of Formulation B were mixed with 990 ul of ABTS reagent and incubated for 2 minutes before the absorbance was measured by UV visible spectrophotometer at 734 nm.

### Animal Model

60 Wistar rats and 60 SHR were divided into six treatment groups separately (excluding the vehicle group). The single blinded experiment was conducted with Formulation B and other drugs being administrated once daily orally (see Table 9). After a week treatment, Wistar rats underwent a MCAO procedure to induce stroke. Treatment was continued for another week after surgery before the rats were sacrificed by decapitation. DNA extraction and DNA base damage analysis was then performed.

### Evaluation of DNA Damage

DNA was extracted from blood samples collected from different treatment groups of Wistar rats and SHR using the phenol-chloroform method. The DNA samples were then analyzed by GC/MS.

### Results:

### Inhibition of Pyrogallol Red Bleaching by Hypochlorous Acid (HOCL)

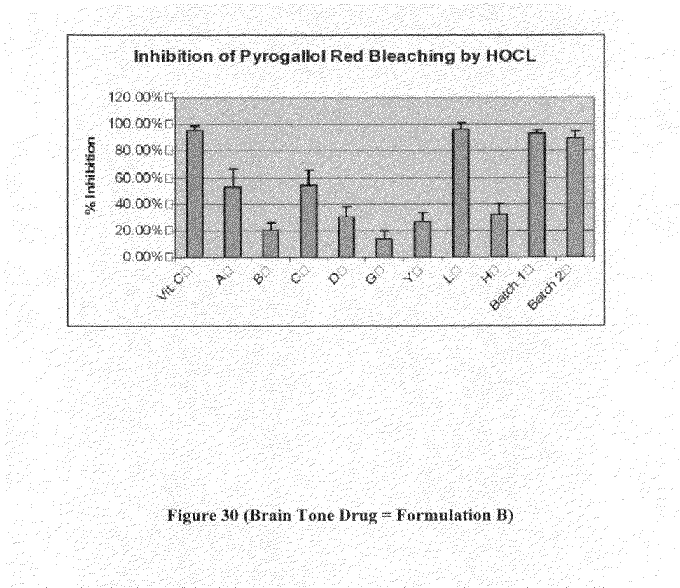

Formulation B (Batch 1 and 2) and Camellia sinensis have pyrogallol red bleaching inhibition effects similar to that of ascorbic acid (˜100%). Other herbal show varying different results from each other. (FIG. 30)

### Inhibition of ABTS Assay

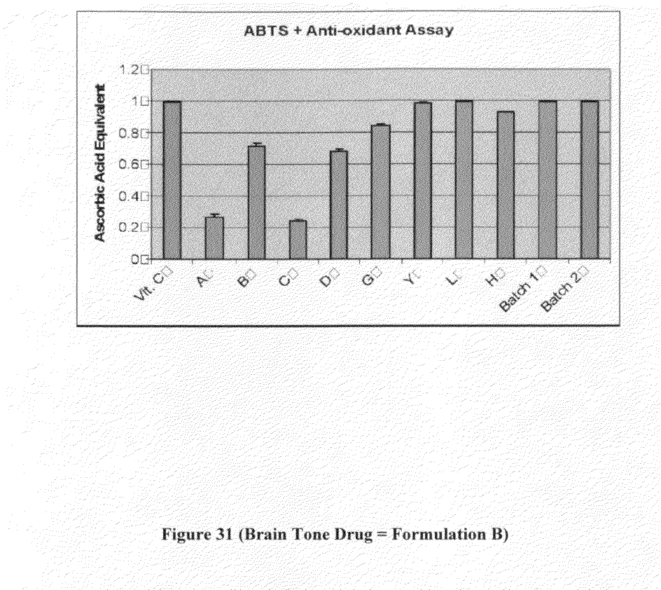

Formulation B (Batch 1) shows it has the same scavenging effect as ascorbic acid (100%), and Formulation B (Batch 2) has a similar scavenging effect of ABST close to that of ascorbic acid (99.67%). Other herbs that have similar effects to ascorbic acid on this assay include Herba leonuri, Camellia sinensis, and Rhodiola rosea (FIG. 31).

### Evaluation of DNA Damage

Formulation B-treated groups show a significantly lower quantity of the oxidized DNA base of FAPy Guanine when compared to the Vehicle group. (Table 10 and 11)

### Conclusions:

The experimental results of the Pyrogallol Red and ABTS assay demonstrate that all the herbs have certain degree of antioxidant capacities. However, they do not show the same trend in antioxidant activities in both tests, because they have different scavenging ability or effects on different radicals (HOCL and ABTS).

In this study, it has been demonstrated that Formulation B has a significantly strong antioxidant effect (almost equal to that of ascorbic acid), which suggests that the antioxidant capacities of the various herbs in Formulation B reinforce each other rather than canceling each other out.

Since Formulation B has strong antioxidant activity, and one of the factors of DNA damage is caused by oxygen free radicals, Formulation B may be preventative of DNA damage. Its antioxidant activity may also be one mechanism by which it works to prevent ischemic damage post stroke.

TABLE 9

| Treatment | Dosage | Number of Rats |
|---|---|---|
| Sham | — | 10 |
| Sham + Formulation B | 500 mg/kg/day | 10 |
| Formulation B | 500 mg/kg/day | 10 |
| Vehicle | 500 mg/kg/day | 10 |
| Ginkgo | 100 mg/kg/day | 10 |
| Losartan | 1 mg/kg/day | 10 |
| Ramipril | 0.5 mg/kg/day | 10 |

TABLE 10
(Braintone = Formulation B)

| | Average | Vehicle | Sham + Braintone | Sham |
|---|---|---|---|---|
| 1 | 5CI Uracil | 0.01 ± 0.01 | 0.11 ± 0.01 | 0.01 ± 0.00 |
| 2 | 5OH, Me Hydantoin | 0.12 ± 0.16 | 0.36 ± 0.19 | 0.13 ± 0.06 |
| 3 | 5Formyl Uracil | 0.17 ± 0.05 | 0.28 ± 0.01 | 0.89 ± 0.65 |
| 4 | 5OH Uracil | 0.01 ± 0.00 | 0.03 ± 0.00 | 0.05 ± 0.03 |
| 5 | 5(OH, Me) Uracil | 0.04 ± 0.01 | 0.01 ± 0.02 | 0.05 ± 0.03 |
| 6 | 5OH Cytosine | 0.01 ± 0.01 | 2.24 ± 0.01 | 0.04 ± 0.02 |
| 7 | Thymine Glycol (cis) | 3.71 ± 3.44 | 7.09 ± 3.30 | 4.88 ± 3.48 |
| 8 | Hypoxanthine | 18.09 ± 19.20 | 4.89 ± 1.74 | 8.09 ± 2.79 |
| 9 | FAPy Adenine | 0.08 ± 0.07 | 0.06 ± 0.05 | 0.12 ± 0.04 |
| 10 | 8OH Adenine | 0.06 ± 0.05 | 1.49 ± 0.02* | 0.06 ± 0.02 |
| 11 | Xanthine | 5.94 ± 3.77 | 1.48 ± 0.86 | 2.52 ± 1.39 |
| 12 | 2OH Adenine | 0.04 ± 0.02 | 0.02 ± 0.01 | 0.02 ± 0.02 |
| 13 | FAPy Guanine | 0.06 ± 0.06 | 0.05 ± 0.01* | 0.02 ± 0.02* |
| 14 | 8OH Guanine | 0.23 ± 0.16 | 0.07 ± 0.01 | 0.12 ± 0.08 |

Oxidized DNA base products were analyzed and quantified by GC/MS and Vehicle group was set as positive control. Results for nonstroke groups Sham+Braintone and Sham are expressed as mean ± S.D. of 3 determinations performed in triplicate and expressed as nmol/mg DNA.
*significantly different from Vehicle group results.

TABLE 11
(Braintone = Formulation B)

| | | Stroke + | Stroke + | Stroke + | Stroke + |
|---|---|---|---|---|---|
| | Average | Braintone | Ginkgo | Ramipril | Losartan |
| 1 | 5CI Uracil | 0.01 ± 0.01 | 0.02 ± 0.01 | 0.01 ± 0.00 | 0.02 ± 0.01 |
| 2 | 5OH, Me Hydantoin | 0.16 ± 0.08 | 0.15 ± 0.06 | 0.04 ± 0.02 | 0.10 ± 0.02 |
| 3 | 5Formyl Uracil | 0.57 ± 0.40 | 0.40 ± 0.21 | 0.50 ± 0.03 | 0.01 ± 0.01 |
| 4 | 5OH Uracil | 0.03 ± 0.02 | 0.02 ± 0.02 | 0.06 ± 0.00* | 0.01 ± 0.01 |
| 5 | 5(OH, Me) Uracil | 0.13 ± 0.07 | 0.03 ± 0.02 | 0.03 ± 0.00 | 0.04 ± 0.03 |
| 6 | 5OH Cytosine | 0.03 ± 0.01 | 0.03 ± 0.01 | 0.05 ± 0.01* | 0.01 ± 0.01 |
| 7 | Thymine Glycol (cis) | 2.99 ± 0.59 | 2.17 ± 0.84 | 0.68 ± 0.31 | 2.20 ± 0.15 |
| 8 | Hypoxanthine | 23.40 ± 8.95 | 16.37 ± 6.29 | 18.12 ± 11.18 | 22.21 ± 8.01 |
| 9 | FAPy Adenine | 0.09 ± 0.02 | 0.10 ± 0.02 | 0.08 ± 0.03 | 0.09 ± 0.03 |
| 10 | 8OH Adenine | 0.05 ± 0.03 | 0.06 ± 0.01 | 0.03 ± 0.01 | 0.05 ± 0.04 |
| 11 | Xanthine | 3.50 ± 1.17 | 3.45 ± 0.94 | 2.61 ± 1.64 | 1.51 ± 0.32 |
| 12 | 2OH Adenine | 0.03 ± 0.02 | 0.02 ± 0.01 | 0.03 ± 0.02 | 0.02 ± 0.03 |
| 13 | FAPy Guanine | 0.03 ± 0.02* | 0.03 ± 0.02* | 0.03 ± 0.01* | 0.03 ± 0.03 |
| 14 | 8OH Guanine | 0.10 ± 0.04 | 0.12 ± 0.03 | 0.13 ± 0.07 | 0.13 ± 0.04 |

Oxidized DNA base products were analyzed and quantified by GC/MS and Vehicle group was set as positive control. Results for stroke treatment groups Stroke+Braintone, Stroke+Ginkgo and Stroke+Losartan are expressed as mean ± S.D. of three determinations
*significantly different from Vehicle group results.

### Example 6

### Anti-Apoptotic Effects of Formulation B in Stroke

### Objectives:

In this study, therapeutic effects of Formulation B on cerebral protection in Wistar rats with MCAO were evaluated. Comparison on the therapeutic effects was also conducted between herbal extract, Ginkgo biloba and two other western medicine: Losartan and Ramipril.

### Methodology:

### Animal Model:

A 105 Wistar rats (220-250 g, male) were randomly divided into 7 treatment groups (In sham groups, rats have sham operation but no MCAO). Formulation B, Ginkgo and 2 other western medicines were administrated orally once daily for 7 days prior to MCAO. After MCAO, the rats were treated with the items continuously for another week (See Table 12). At the end of the treatment (day 14), the animals were sacrificed. The brains were collected for further gene expression studies.

### RNA Isolation:

The cerebral left cortexes containing infarct area were separated. Three left cortexes from rats in each treatment groups were taken for RNA isolation and subsequent Reverse-transcriptase Polymerase Chain Reaction (RT-PCR). The brain samples were homogenized in iced TRIzol reagent using a Polytron homogenizer. RNA was extracted through two consecutive ethanol precipitations separated by an additional phenol extraction step.

### RT-PCR:

After standardization of Glyceraldehydes-3-phosphatase dehydrogenase (GAPDH), the corresponding volumes of RNA from each treatment group were used for RT-PCR by using the Qiagen One-step RT-PCR kit. Primers sequences were used for each gene used in current study are shown in Table 13.

### Immunohistochemical Staining Using Frozen Sections:

The localization of the protein products of targeted genes was identified by immunohistochemical staining. The brains (n=3) from each treatment group were collected and post-fixed with 2% paraformaldehyde for 2 hours and then transferred into 25% sucrose in phosphate buffer for 2 days (max.) for dehydration. The brains were than kept in −20 deg.C. phosphate buffered saline for further use.

Brain tissues were cut at 20 um using Leica CM1510 cryostat. The sections were then fixed on the polysine-coated slides. HRP/DAB kit was used for antibody staining. The brain tissue sections were incubated with primary antibody for 3 hours. Polyclonal rabbit anti-AT2 antibody, polyclonal rabbit anti-Bax antibody, polyclonal rabbit anti-FAS antibody and polyclonal rabbit anti-Bcl-xS/xL antibody were used for the primary incubation. After the primary incubation, the tissue sections were incubated with Biotinylated Goat Anti-Polyvalent for an hour at room temperature. Subsequently, slides were incubated with streptavidin peroxidase which can conjugate with biotin present on the secondary antibody for 10 minutes. Colorimetric detection was performed with 3,3′-diaminobenzidine tetrahydrochloride (DAB) working solution to detect the specific antibody, secondary antibody and streptavidin-enzyme complex.

For counterstaining, slides were rinsed in hematoxylin for 4 times and there were washed in running tap water immediately to remove all traces of hematoxylin from the sections. Lastly, the tissue sections were mounted by a drop of mounting medium permount by coverslip. Slides were viewed using a fluorescent microscope.

A fluorometric TUNEL system kit was used for detection of apoptosis-induced nuclear DNA fragmentation via fluorescence microscopy.

### Results:

### Gene Expression:

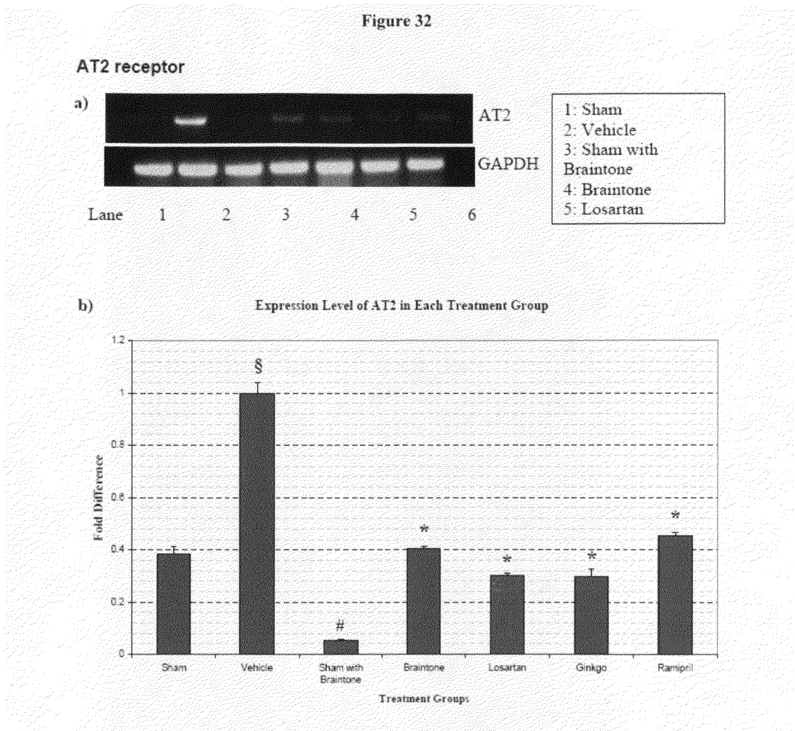

AT2 receptor (FIG. 32 a & b): Formulation B had significantly reduced the expression level of AT2 expression level in the sham operate groups by 0.05 fold compared with the sham treated group (p<0.05), and, Formulation B along with Losartan, gingko and Ramipril also significantly reduced the AT2 expression in stroke operated groups compared with the stroke operated vehicle group (p<0.05).

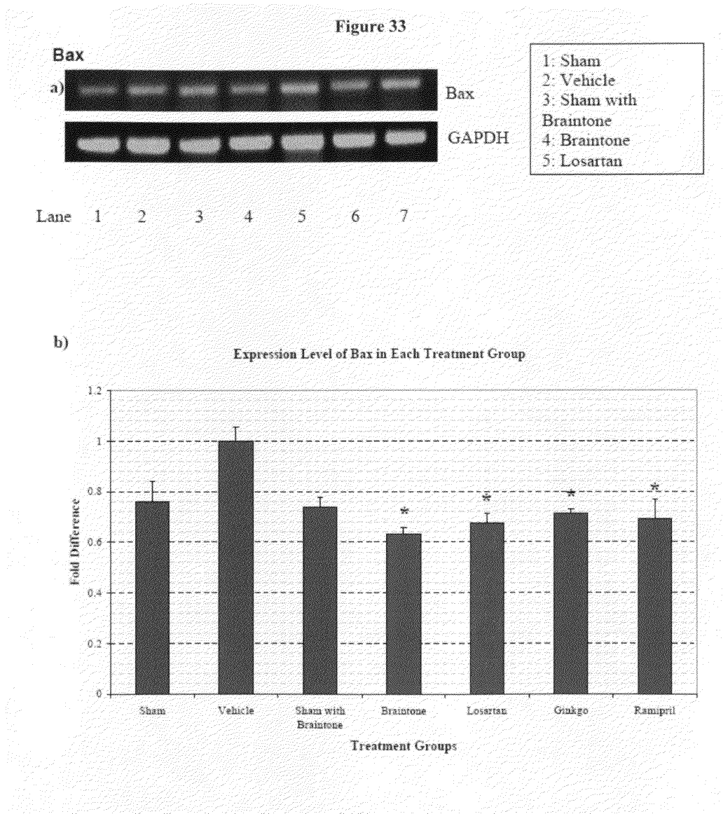

Bax (FIGS. 33 a & b): Formulation B along with Losartan, gingko and Ramipril had significant effect in down regulating Bax gene expression in stroke-operated group (0.63 fold) compared with the stroke operated vehicle group (p<0.05).

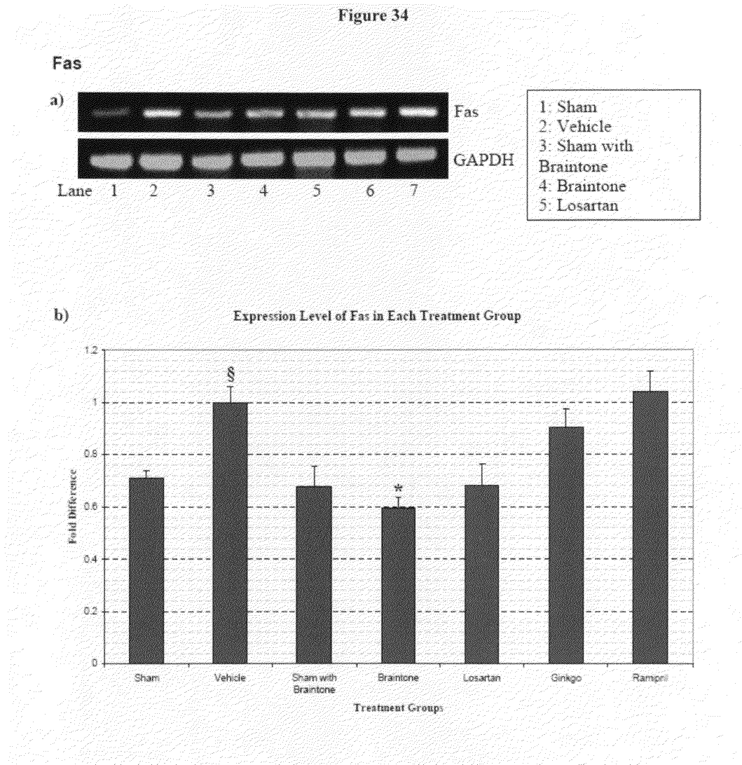

Fas (FIGS. 34 a & b): Formulation B could significantly down regulate Fas expression (0.62 fold) in stroked-operated group compared with the stroke operated vehicle group (p<0.05). In contrast, Gingko, Ramipril and Losartan have no significant effect on Fas expression in stroke-operated group compared with the stroke operated vehicle group.

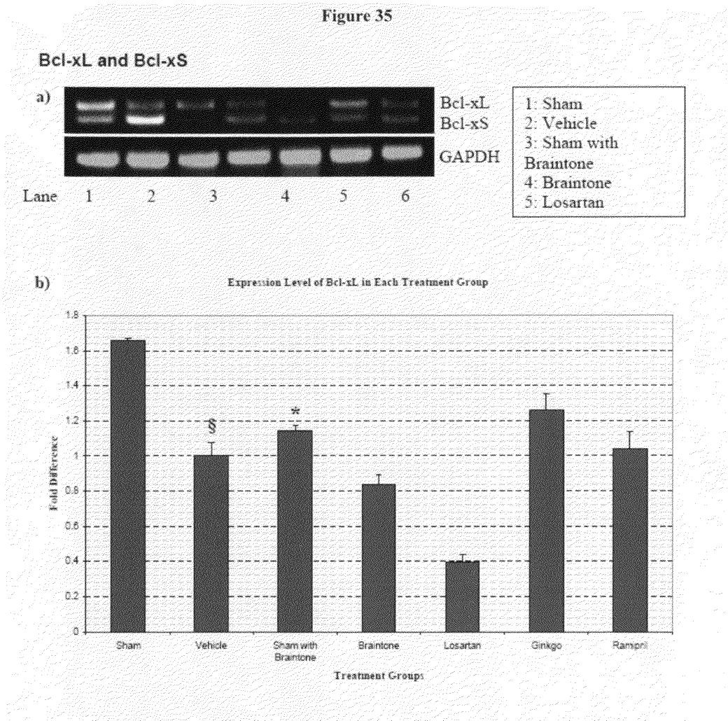

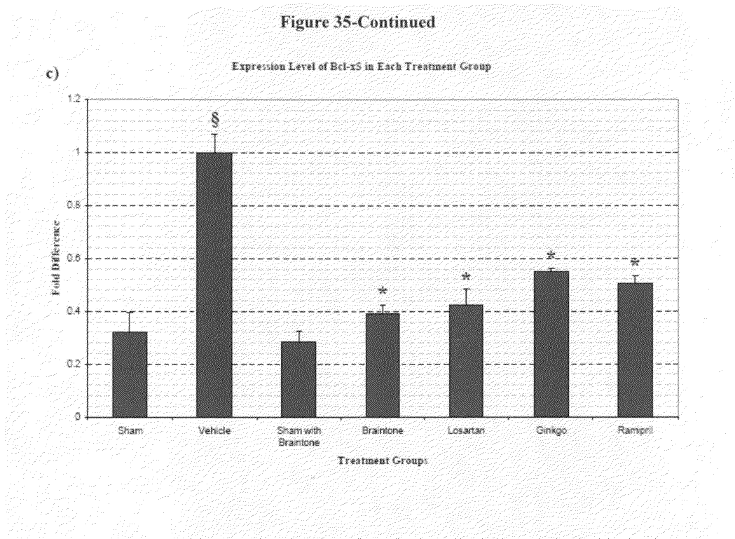

BcL-xL (FIG. 35, a, b): BcL-xL expression statistically decreased in stroke operated vehicle groups as compared to sham groups. Formulation B had decreased the expression level of BcL-xL compared to the stroke operated vehicle group at 0.84 fold although the decrease was not statistically significant. Losartan, gingko and Ramipril also did not have any significant effects on up or down regulation of this gene. Formulation B also showed significantly reduced expression of this gene in the sham operated group treated with Formulation B compared with the sham operated group (p<0.05). It's unknown why Formulation B down regulated this anti-apoptotic gene.

BcL-xS (FIG. 35 a, c): BcL-xS expression significantly increased in stroked rats as compared to the sham groups. Formulation B did not have significant effect on BcL-xS in the sham-operated group, but significantly reduced expression of BcL-xS when compared with the stroke operated vehicle group like Losartan, gingko and Ramipril did (p<0.05).

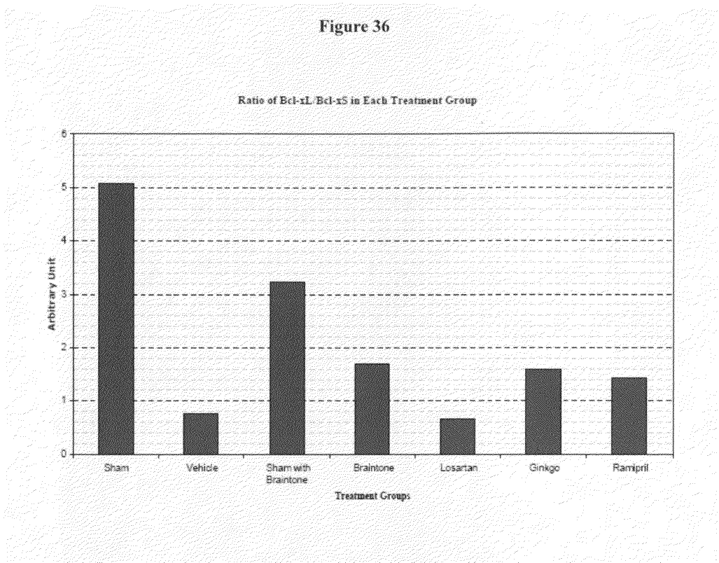

Ratio of BcL-xL and BcL-xS (FIG. 36): At a ratio>1, cells are likely in a anti-apoptotic state whereas a ratio of <1, indicates cells in a pro-apoptotic state. Formulation B-treated sham and vehicle groups were in anti-apoptotic states with ratios 3.2 and 1.7 respectively.

### Conclusion:

Results of gene expression give strong evidence that over-expression of pro-apoptotic (AT2 receptor, Bax, Fas and Bcl-xs expression level↑) and down-regulation of anti-apoptotic genes (Bcl-xl expression level↓) occurs after MCAO.

This study shows that Formulation B could significantly reduce the AT2 and Bax expression (by 0.4 and 0.63 fold respectively), suggesting Formulation B can interfere with apoptosis and thereby potentially reduce the infarct area post ischemic event.

It is well documented that up regulation of Fas is involved in ischemic brain injury. Formulation B could significantly reduce the Fas as compared to other treatment groups including gingko, further demonstrating Formulation B has a promising therapeutic potential for ischemic stroke. One mechanism by which Formulation B may work is as an antioxidant to scavenge the free radicals and inhibit free radical generation. In so doing it may inhibit the free radical mediated extrinsic and intrinsic apoptosis signaling pathways.

Bcl-x has 2 isoforms; Bcl-xl facilities cell survival, whereas Bcl-xs is a pro-apoptotic protein. In this study, Bcl-xl was significantly reduced and Bcl-xs was significantly increased after MCAO when compared with the sham-operated group. Down-regulation of Bcl-xl coupled with up-regulation of Bcl-xs could serve as an accelerator of apoptotic processes after MCAO. Although it is unknown why Formulation B could also reduce Bcl-xl expression, it maintained the ratio of Bcl-xl/Bcl-xs>1 in stoke-operated group which was originally <1 in the vehicle stroke operated group.

Though Ginkgo is a component of Formulation B, it did not perform better than Formulation B on down regulating Fas suggesting it may not have a similar therapeutic effect for stroke treatment as compared with Formulation B. Therefore, the synergistic effects of Formulation B, acting as an antioxidant may be more beneficial in preventing stroke damage.

TABLE 12
(Braintone = Formulation B)
Dosage of each drug.

| Treatments | Dosage | Number of Rats |
|---|---|---|
| Sham | Nil | 15 |
| Vehicle | water | 15 |
| Sham with Braintone | 500 mg/kg/day | 15 |
| Braintone | 500 mg/kg/day | 15 |
| Losartan | 1 mg/kg/day | 15 |
| Ginkgo | 50 mg/kg/day | 15 |
| Ramipril | 0.1 mg/kg/day | 15 |

TABLE 13

Primer sequences of GAPDH, AT2, Bax, Fas, Bcl-xL
and Bcl-xS used in current studies

| | | Base Pairs & |
|---|---|---|
| Primers | Sequences | function |
| GADPH sense | 5′-CATGGTCTACATGTTCCAGT-3′ | 349 bp |
| GADPH antisense | 5′-GGCTAAGCAGTTGGTGGTGC-3′ | House keeping |
| AT2 sense | 5′-TTGCTGCCACCAGCAGAAAC-3′ | 1179 bp |
| AT2 antisense | 5′-GTGTGGGCCTCCAAACCATTGCTA-3′ | Apoptosis |
| Bax sense | 5′-GCAGGGAGGATGGCTGGGGAGA-3′ | 579 bp |
| Bax antisense | 5′-TCCAGACAAGCAGCCGCTCACG-3′ | Apoptosis |
| Fas sense | 5′-AACATGAGAACATCCTGTGCC-3′ | 251 bp |
| Fas antisense | 5′-TCCCTGCTCATGATGTCTACC-3′ | Apoptosis |
| Bcl-xL sense | 5′-AAAATGTCTCAGAGCAACCGG-3′ | 702 bp |
| Bci-xL antisense | 5′-TCACTTCCGACTGAAGAGTGA-3′ | Anti-apoptosis |
| Bcl-xS sense | 5′-AAAATGTCTCAGAGCAACCGG-3′ | 510 bp |
| Bcl-xS antisense | 5′-TCACTTCCGACTGAAGAGTGA-3′ | Pro-apoptosis |

### Example 7

### Effect of Remembrance (Formulation A) on Ischemic Stroke Rat Model: a Chronic Study

### Objective:

This study evaluated and investigated the effect of chronic treatment of the inventive composition of Remembrance (Formulation A) compared to well-known western drugs and other substances in spontaneous hypertension rats (SHR) and Wistar rats with stroke induced middle cerebral artery occlusion (MCAO). It also evaluated the role of Remembrance in the cerebral protection and treatment.

### Methodology and Results:

96 Wistar rats and 96 SHR, weighing 250-300 g, were randomly divided into eight treatment groups respectively (see Table 14), Remembrance (Formulation A) and other comparative drugs were dissolved in distilled water and administrated orally once daily for two months (Table 14: dosage of administration). Then stroke was induced by occlusion of middle cerebral artery (MCA). Remembrance and the drugs were continued for another month. At the end of treatment period, all rats were scarified by decapitation after measuring the hemodynamic parameters. Brains and livers were collected for further studies.

TABLE 14
Dosage of administration

| Treatment | Dosage | Number of Rats |
|---|---|---|
| Sham | — | 12 |
| Sham + Remembrance | 500 mg/kg/day | 12 |
| Vehicle | 4 ml/kg/day | 12 |
| Remembrance | 250 mg/kg/day | 12 |
| Remembrance | 500 mg/kg/day | 12 |
| Ginkgo | 20 mg/kg/day | 12 |
| Aspirin | 10 mg/kg/day | 12 |
| Losartan | 1 mg/kg/day | 12 |

N = 96 Wistar Rats N = 96 Spontaneous Hypertension Rats (SHR)

### Study Timeline:

Oral administration of Remembrance/other treatments (2 months)→MACO→Continue the corresponding treatment after MACO (1 month)→Measurements

Mortality Rate: (The mortality rates of the rats with ischemic stroke in different treatment groups were compared)

TABLE 15
Number of Wistar Rat died after MACO

| Groups | # of Wistar Rats died | Total # of Wistar Rats |
|---|---|---|
| Sham | 0 | 8 |
| Sham + Remembrance | 0 | 9 |
| Vehicle | 5 | 12 |
| Remembrance (250 mg) | 3 | 9 |
| Remembrance (500 mg) | 2 | 9 |
| Ginkgo | 2 | 8 |
| Aspirin | 2 | 5 |
| Losartan | 1 | 8 |

As shown in Table 15, relatively less Wistar rats died in Remembrance-treated groups.

TABLE 16
Number of SHR died after MACO

| Groups | # of SHR Rats died | Total # of SHR Rats |
|---|---|---|
| Sham | 0 | 12 |
| Sham + Remembrance | 0 | 9 |
| Vehicle | 4 | 13 |
| Remembrance (250 mg) | 5 | 11 |
| Remembrance (500 mg) | 1 | 9 |
| Ginkgo | 5 | 12 |
| Aspirin | 4 | 11 |
| Losartan | 3 | 9 |

As shown in Table 16, relatively less SHR died in Remembrance-treated (500 mg) group.

### Clinical Evaluation of Ischemia by Neurological Scores:

A neurological scale of 0 to 5 was used to assess the motor and behavioral changes observed in the ischemic rats after the MACO procedure. The evaluation scale is summarized in Table 17.

TABLE 17
Neurological evaluation of rats after MACO

| Score | Evaluation |
|---|---|
| 0 | No apparent deficit |
| 1 | Failure to extend contralateral forepaw fully |
| 2 | Spontaneous movement in all directions; contralateral |
| | circling only if pulled by tail |
| 3 | Spontaneous contralateral circling |
| 4 | Inactive, walk only when stimulated |
| 5 | Dead |

TABLE 18
Neurological Scores of Wistar Rats (Braintone = Rembrance = Formulation A)

| | 1 day after | 15 days after | 1 day before |
|---|---|---|---|
| Group (Wistar) | stroke | stroke | decapitation |
| Sham n = 8 | 0 | 0 | 0 |
| Sham + Brainstone n = 9 | 0 | 0 | 0 |
| Vehicle n = 12 | 1.47 ± 1.68 †† | 2 ± 2.27 †† | 1.93 ± 2.31 † |
| Braintone (250 mg/kg) n = 9 | 1.56 ± 1.33 | 1.22 ± 2.17 | 1.78 ± 2.44 |
| Braintone (500 mg/kg) n = 9 | 0.45 ± 1.51 | 1.33 ± 2.04 | 1.11 ± 2.20 |
| Ginkgo n = 8 | 1 ± 1.77 | 1.4 ± 2.26 | 1.5 ± 2.20 |
| Aspirin n = 5 | 1 ± 1.73 | 2 ± 2.45 | 2 ± 2.74 |
| Losartan n = 8 | 0 | 0.56 ± 1.77 | 0.625 ± 1.77 |

Data is presented as means +/− SD;
† P < 0.05 when compared to Sham group;
†† p < 0.01 when compared to sham group.

As shown in Table 18, Remembrance-treated groups (250 and 500 mg/kg/day) had lower neurological scores compared to other treatment groups (Aspirin-treatment and Vehicle) on the 15th day after MCAO in Wistar Rats. (Low neurological score means less damage of neuron cells).

TABLE 19
Neurological scores of SHR Rats
(Braintone = Remembrance = Formulation A)

| | 1 day after | 15 days after | 1 day before |
|---|---|---|---|
| Group (SHR) | stroke | stroke | decapitation |
| Sham n = 12 | 0 | 0 | 0 |
| Sham + Brainstone n = 9 | 0 | 0 | 0 |
| Vehicle n = 13 | 1.08 ± 1.38 | 1.38 ± 2.10 | 1.69 ± 2.32 |
| Braintone (250) n = 11 | 1 ± 1.18 | 2 ± 2.4 | 2.55 ± 2.38 |
| Braintone (500) n = 9 | 0.56 ± 0.53 | 0.89 ± 1.62 | 0.77 ± 1.64 |
| Ginkgo n = 12 | 1.91 ± 1.68 | 1.92 ± 2.21 | 2.25 ± 2.35 |
| Aspirin n = 11 | 1.72 ± 1.73 | 0.63 ± 1.84 | 1.54 ± 2.15 |
| Losartan n = 10 | 1.67 ± 2 | 1.89 ± 2.23 | 1.89 ± 2.23 |

Data is presented as means +/− SD

As shown in Table 19, Remembrance-treated groups (500 mg/kg/day) also had generally lower neurological scores compared to other treatment groups after MCAO in SHR rats. (Low neurological score means less damage of neuron cells).

### Infarct Volume Assessment:

The area of cerebral infarction was quantified with TTC (2,3,5-triphenyltetrazolium chloride) staining. The cerebrum was removed and cleared of all overlying membranes. It was sectioned into 8 pieces of 2-mm thick coronal slices, and then stained with 0.1% TTC solution at 37 deg. C. for 30 minutes. Healthy brain tissue would turn purple on staining while the infarct area due to ischemia left a white unstained area. The infarct size would be analyzed by an image analyzer system (Scion image for windows, Beta 4.0.2) and converted by integration to the true infarct size of ischemia damage.

Wistar Rats

| | | | |
|---|---|---|---|
| Sham (W.S): n = 3 | Sham + Remembrance | Vehicle (W.Veh): n = 3 | 250 mg Remembrance |
| | (W.S + BT): n = 3 | | (W.BT 250): n = 2 |
| 500 mg Remembrance | Ginkgo (W.G): n = 2 | Aspirin (W.A): n = 1 | Losartan (W.L): n = 2 |
| (W.BT 500): n = 2 | | | |

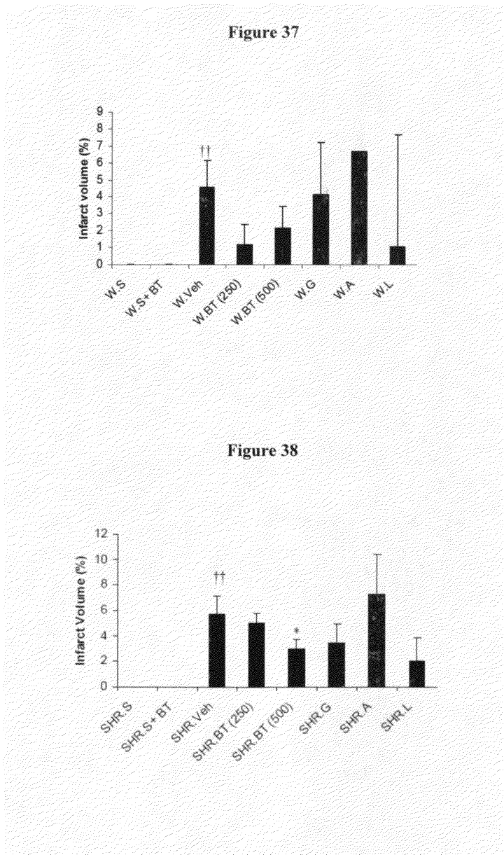

As shown in FIG. 37, Remembrance-treated groups (250 and 500 mg/kg/day, n=2) had lower (but not significant) infarct volume compared to the other treatment groups (Vehicle and Aspirin-treatment groups).

SHR Rats

| | | | |
|---|---|---|---|
| Sham (SHR.S): n = 3 | Sham + Remembrance | Vehicle (SHR.Veh): n = 3 | 250 mg Remembrance |
| | (SHR.S + BT): n = 3 | | (SHR.BT 250): n = 2 |
| 500 mg Remembrance | Ginkgo (SHR.G): n = 2 | Aspirin (SHR.A): n = 2 | Losartan (SHR.L): n = 2 |
| (SHR.BT 500): n = 3 | | | |

As shown in FIG. 38, Remembrance-treated groups (500 mg/kg/day, n=3) had significantly lower infarct volume compared to the vehicle group. (p<0.05 when compared to vehicle)

### Brain Capillary Density and Morphology:

The differentiation of coronary capillaries and evaluation of morphological changes after cerebral ischemia were performed using Hematoxylin and Eosin Staining. Random slides of the rat brains were used for the staining.

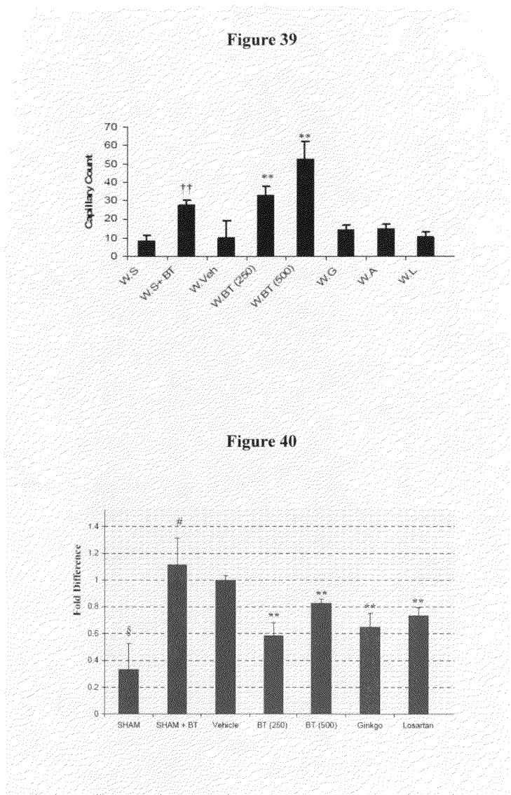

As shown in FIG. 39, Remembrance-treated groups (250 and 500 mg/kg/day, n=3 for each group) had significantly high capillary count in Wistar rats (p<0.01 when compared to Vehicle)

### Gene Expression:

Glyceraldehydes-3-phosphatase dehydrogenase (GAPDH) was used as the internal standard gene. Primers sequences of GAPDH, VEGF, eNOS, ACE, AT1, AT2, Fas, Bax, Bcl-xL and Bcl-xS were used in this study.

AT2 receptor is a factor of neurological cell loss and mediates apoptosis. Effects of AT2 receptor include inhibition of cell growth, fetal tissue development, modulation of extra-cellular matrix, apoptosis. Down-regulation of AT2 receptor expression can prevent the onset of apoptosis and therefore enhance neuro-protection after ischemic injury.

As shown in FIG. 40, Remembrance-treated group (250 and 500 mg/kg/day) showed a significant fold decrease of AT2 receptor expression in Wistar rats compared to vehicle (MCAO rat w/o treatment) group. (p<0.01)

SHR (n=3)

As shown in FIG. 41, No significant difference between Remembrance-treated groups and Vehicle group in SHR.

BAX is a pro-apoptotic gene, down-regulation of this gene after ischemic brain injury is desirable for preserving neuronal cells.

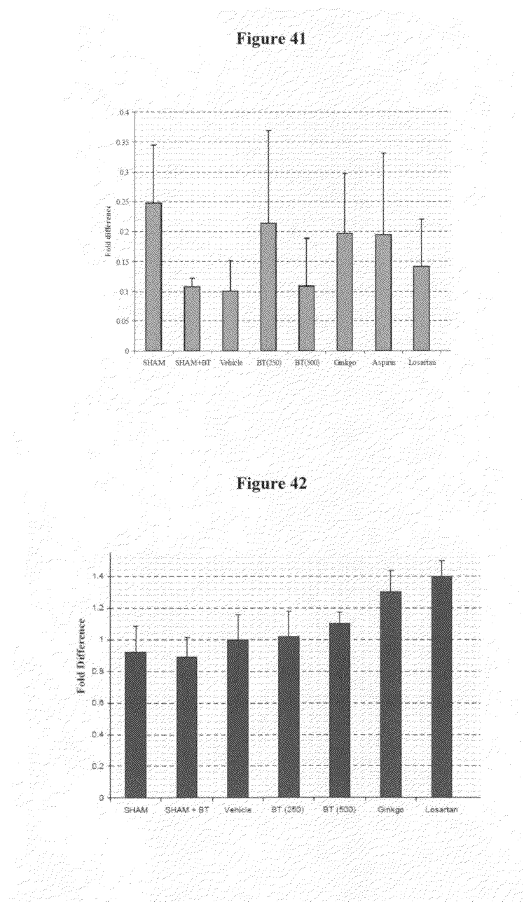

As shown in FIG. 42. No significant difference between Remembrance-treated groups and Vehicle group in Wistar rats.

SHR (n=3)

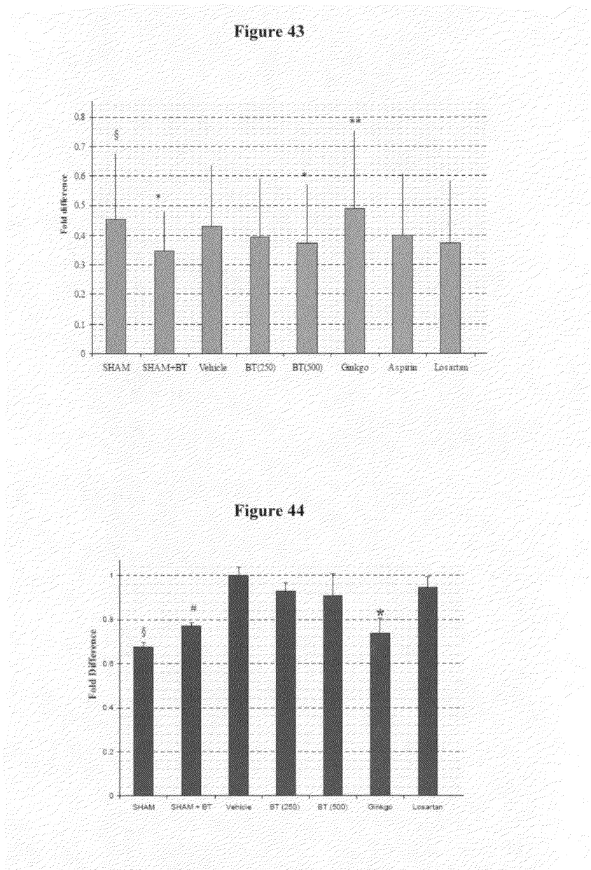

As shown in FIG. 43, Remembrance-treated groups generally decrease the BAX expression, it is significant in 500 mg Remembrance group compared to vehicle (MCAO rat w/o treatment) group (p<0.05) and in the Sham BT compared to vehicle (p<0.05).

This suggests that Remembrance at high dose can interfere with apoptosis and thereby promote the neuro-protection after ischemic brain injury.

FAS gene triggers apoptosis of immune cells. It is well documented that up regulation of FAS is involved in ischemic brain injury. Down-regulation of FAS may be desirable in mitigating damage from ischemic brain injury.

As shown in FIG. 44, Remembrance (both dosages) has a lowering effect of Fas expression, but not statistically significant. No significant difference of FAS expression between Remembrance-treated groups and Vehicle group in Wistar rats.

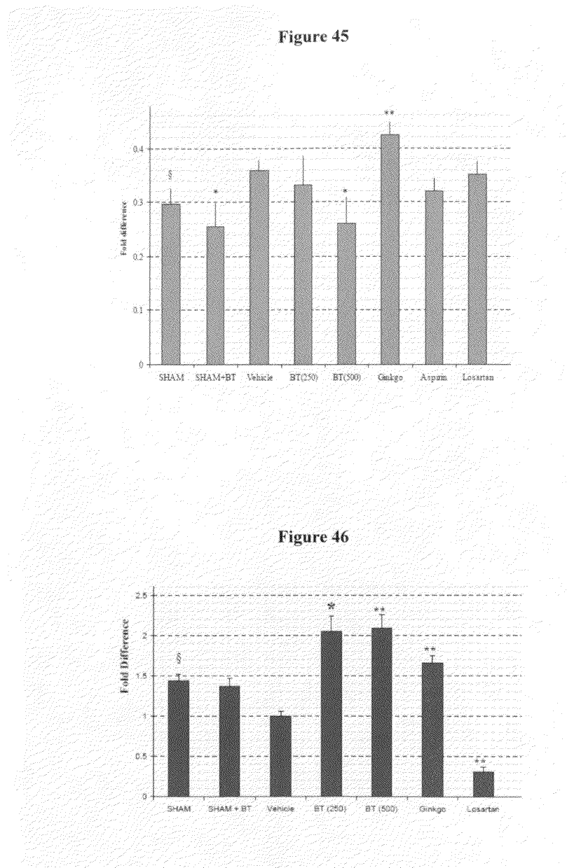

As shown in FIG. 45, FAS expression was up-regulated in vehicle group compared to sham group. Remembrance-treated groups generally decrease the FAS expression. Remembrance (500 mg/kg/day) significantly decreased FAS expression in SHR when compared to vehicle group (p<0.05).

These results show that Remembrance could significantly reduce the FAS expression in spontaneously hypertension rats at a high dose, indicating it may have a promising therapeutic potential for ischemic brain injury.

Bcl-xL facilitates cell survival. Up-regulation of this gene is desirable after MACO injury for neuronal cell protection.

As shown in FIG. 46, Vehicle group has significantly decreased expression compared to sham group. Remembrance (both dosages) in Wistar rats showed significant fold increase compared to vehicle group (2.05 (p<0.05) and 2.09 (p<0.01) respectively). While Losartan group showed a significant fold decrease compared with vehicle group (p<0.01).

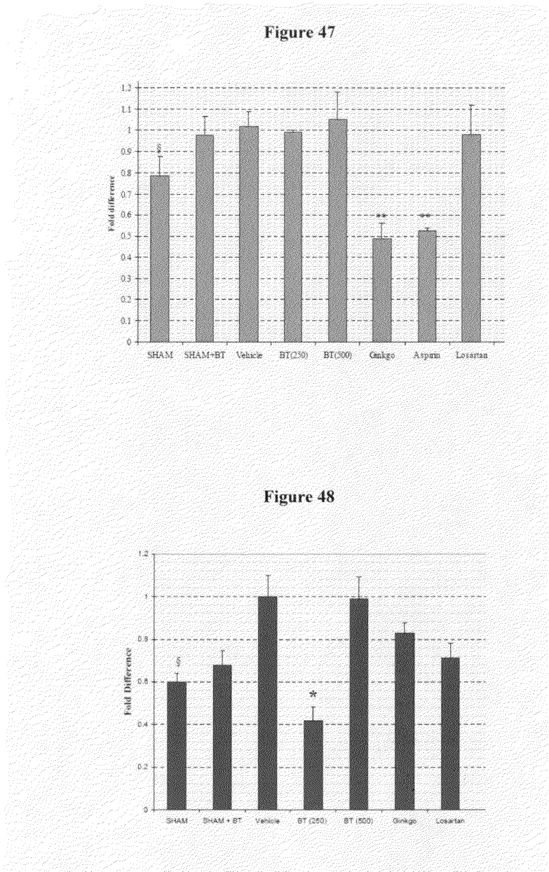

As shown in FIG. 47, No significant difference between Remembrance-treated groups and Vehicle group in SHR.

Bcl-xL facilitates cell survival. It was significantly up-regulated by Remembrance (250 mg and 500 mg) in Wistar rats (but not in SHR) suggesting that Remembrance can promote the brain cell survival (reduce apoptosis) and enhance neuro-protection in Wistar rats.

Bcl-xS is a pro-apoptotic gene. Down-regulation of this gene is desirable after MCAO injury for neuronal cell protection.

As shown in FIG. 48 for Wistar rats, Bcl-xS is significantly increased in stroke-induced rats compared to the sham group. There is no effect on Bcl-xS at 500 mg/kg/day remembrance but significantly reduce the Bcl-xS expression at lower dose (250 mg/kg/day) compared to vehicle group (p<0.05).

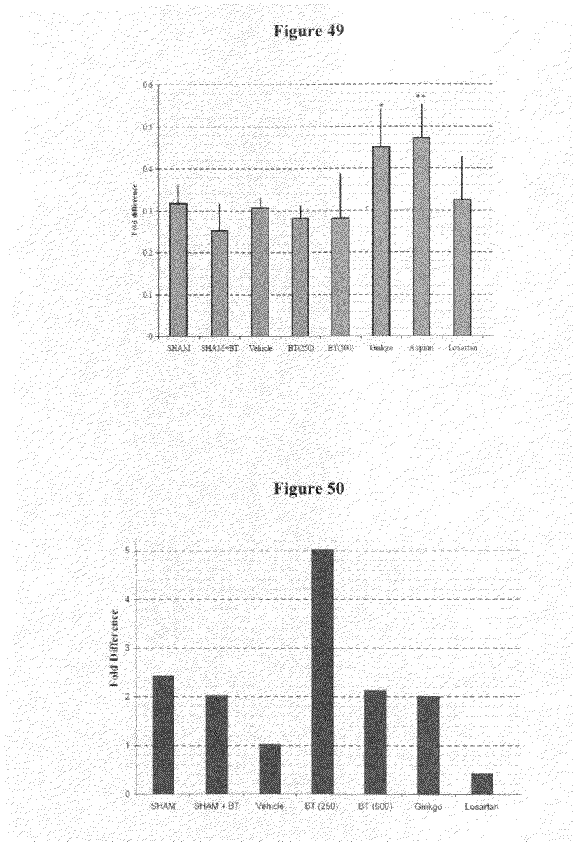

As shown in FIG. 49, No significant difference between Remembrance-treated groups and Vehicle group in SHR.

Bcl-xS is a pro-apoptotic gene which promotes apoptosis. Down-regulation of Bcl-xS under the effect of Remembrance (250 mg) in Wistar rats suggest that low dose of Remembrance can inhibit apoptosis and hence promote neuro-protection. No effect was observed in SHR rats however ginkgo and losartan had up-regulating effects which would be undesirable in the face of neuronal injury.

Bcl-xL/Bcl-xS Ratio

Bcl-x has two isoforms: Bcl-xL facilities cell survival whereas Bcl-xS is a pro-apoptotic protein. Down-regulation of Bcl-xL coupled with up-regulation of Bcl-xS could serve as an accelerator of apoptosis after ischemic stroke. Therefore, Bcl-xL and Bcl-xS ratio>1 indicates the brain cells are in anti-apoptotic state which is essential for neuro-protection after stroke.

As shown in FIG. 50, Remembrance-treated groups (both dosages) are situated in anti-apoptotic state. (Bcl-xL/Bcl-xS ratio>1) compared with the vehicle group in Wistar rats.

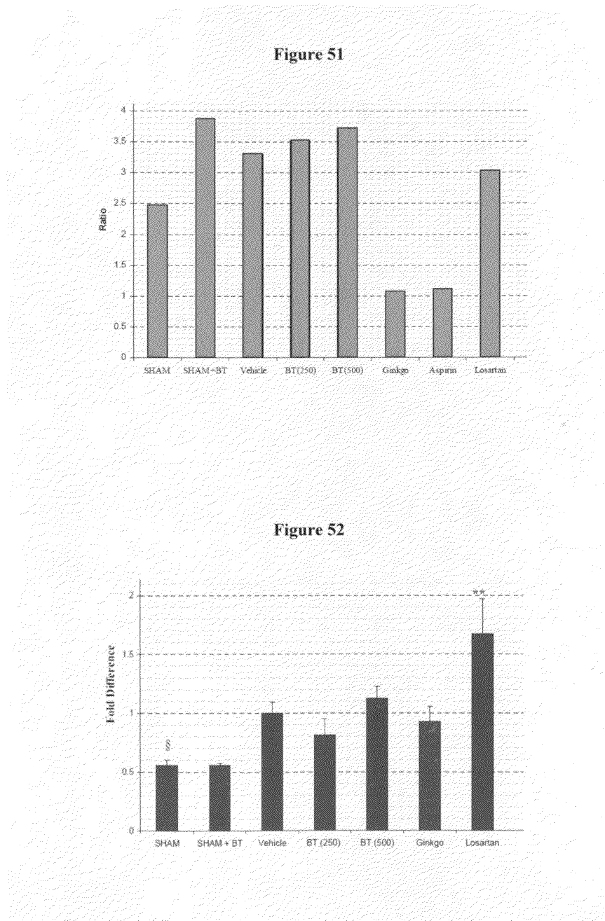

As shown in FIG. 51, No significant difference between Remembrance-treated groups and Vehicle group in SHR.

AT1 receptor: Lowering AT1 expression would result in less AT1 receptors which help prevent hypertension.

As shown in FIG. 52, No significant difference between Remembrance-treated groups and Vehicle group in Wistar rats.

As shown in FIG. 53, AT1 expression was up-regulated in vehicle group. Remembrance-treated groups (both dosages) generally down-regulate the AT1 expression in SHR.

Remembrance appears to lower the gene expression of AT1 in SHR and thus suggest a role in hypertension prevention.

eNOS gene results in the production of nitric oxide. Nitric Oxide (NO) produced in the endothelial cells is involved in vasorelaxation and mechanisms of cardiovascular homeostasis. Up-regulation of this gene is favorable in generating NO in blood vessels which is desirable for vascular function and angiogenesis.

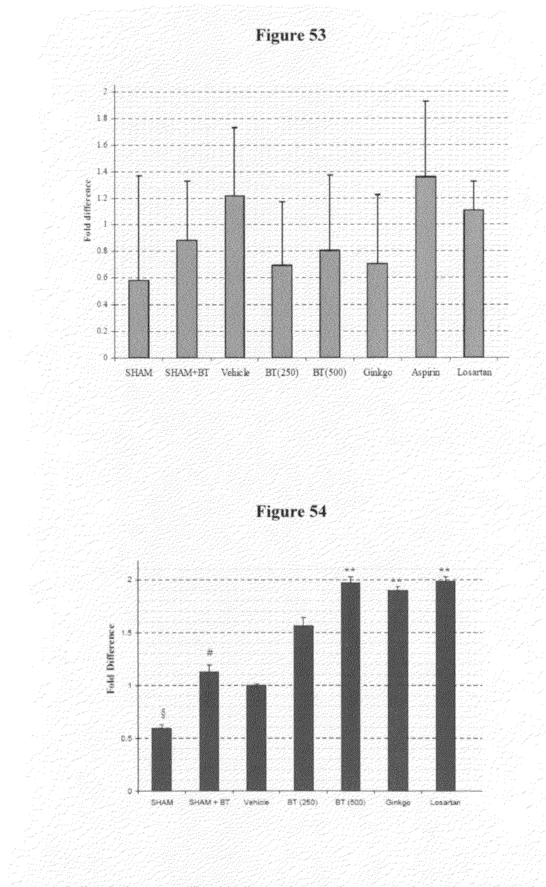

As shown in FIG. 54, eNOS expression has statistically increased in vehicle group compared to sham group in Wistar rats. Remembrance (500 mg/kg/day) significantly increases the eNOS level compared to vehicle (p<0.01).

These results show that Remembrance (500 mg/kg/day) significantly increases the eNOS level compared to vehicle in Wistar rats, implying that NO production was partially restored by Remembrance after the stroke induction, possibly to regulate angiogenesis.

### Apoptosis Assay:

TUNEL staining was used for detection of apoptosis-induced nuclear DNA fragmentation via fluorescence microscopy.

### Wistar Rats:

Apoptotic cells were identified by using TUNEL staining. Apoptosis (GREEN) was detected in the infarct area (BLACK) of left cerebral cortex while no apoptosis was detected in non-infarct area (BLUE) in all groups. Strongest apoptosis marker (GREEN) was observed in vehicle group, while Remembrance-treated group has a reduction of apoptosis marker (Data not shown.).

Apoptotic staining in cerebral cortex after 1 month of MCAO in Wistar rats (40* magnification) for each treatment group.

Remembrance, Aspirin, Losartan and Ginkgo treatment groups have reduction in apoptosis (GREEN). Remembrance treatment groups (250 and 500 mg) have larger reduction of apoptosis marker compared to Aspirin, Losartan and Ginkgo groups.

### SHR

Apoptotic cells were identified by using TUNEL staining. Apoptosis (GREEN) was detected in the infarct area (BLACK) of left cerebral cortex while no apoptosis was detected in non-infarct area (BLUE) in all groups. Strongest apoptosis marker (GREEN) was observed in vehicle group, while Remembrance-treated group has a reduction of apoptosis marker (Data not shown.).

Remembrance, Aspirin, Losartan and Ginkgo treatment groups have reduction in apoptosis (GREEN). Remembrance treatment group (500 mg) has larger reduction of apoptosis marker compared to Aspirin, Losartan and Ginkgo groups.

### DNA Damage:

Wistar rats

| | | | |
|---|---|---|---|
| Sham (WS) | Sham + Remembrance | Vehicle (W.V) | 250 mg Remembrance |
| | (W.S + BT) | | (WBT 250) |
| 500 mg Remembrance | Ginkgo (WG) | Aspirin (WA) | Losartan (WL) |
| (WBT 500) | | | |

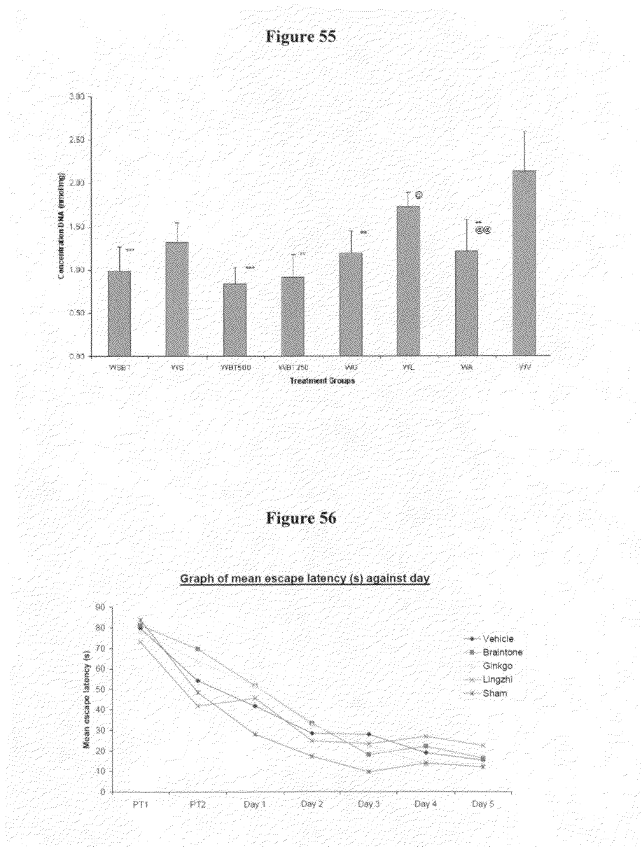

As shown in FIG. 55, The sham+Remembrance group in (***WSBT), stroke-induced Remembrance groups (***WBT500, **WBT250) showed a significantly lower amount of oxidized DNA when compared to Vehicle group (***p<0.0001, **p<0.001)

The stroke-induced Aspirin and Losartan group (WL, WA) showed a higher amount of DNA damage than WBT500 group.

### Discussion:

The brain is the major part of central nervous system; it controls many important activities like thoughts, memory, cognitive and other vital physiological activities such as heart rate and respiration. Since all activities are performed by brain cells, their health is very important in maintaining our body's functions. However, brain cells are sensitive cells and susceptible to damage when there is poor circulation supplying oxygen and nutrients.

The experimental results show the infarct volume after MCAO in Wistar rats' brains appears lower in Remembrance-treated groups (250 and 500 mg/kg/day) in Wistar rats and significantly lower in the 500 mg/kg/day Remembrance-treated group compared to vehicle in SHR, which indicates that Remembrance may reduce the area of tissue necrosis and apoptosis associated with ischemia. Remembrance-treated groups, especially at 500 mg/kg/day for both Wistar and SHR rats had lower neurological scores compared to the vehicle suggesting Remembrance is associated with a better neurological outcome after an ischemic event. Additionally, Remembrance (500 mg/kg/day) had a relatively smaller number of dead rats when compared to ginkgo, aspirin, losartan or the control vehicle group after MCAO in SHR rats. Capillary density was also significantly increased in all Wistar rats treated with Remembrance including the sham group. These results suggest that Remembrance may better preserve neurological function and blood circulation in the face of an ischemic event compared to ginkgo or aspirin alone. Good circulation of blood and nutrients to the brain ensures the continued health of neuron cells, and therefore helps maintain brain cognitive and memory functions. One mechanism by which Remembrance may work to improve neurological outcome in the face of an ischemic event is to increase capillary density.

During an ischemic stroke, complex chemical and electrical processes lead to the death of nerve cells. Other ways in which Remembrance may work is to provide neuro-protection by modulating gene expression to minimize the damage that results when brain cells are deprived of oxygen and nutrients. From the gene expression results, we can classify the genes into 2 categories: genes that are involved in apoptosis and genes that affect vascular re-adjustment.

Down regulation of AT₂, Bax, Fas, Bcl-Xs is desirable for preventing apoptosis of neuronal cells. Down regulation in Wistar rats of AT₂ at Remembrance dosages of 250 and 500 mg/kg/day and in Bcl-Xs at 250 mg/kg/day was significant compared to the vehicle. In SHR rats, significant down regulation of Bax and Fas compared to the vehicle was observed at 500 mg/kg/day. Upregulation of Bcl-xl, which facilitates cell survival, was significant in Wistar rats when compared to the vehicle at both 250 and 500 mg/kg/day. In the apoptosis assays, Remembrance also displayed less apoptosis markers compared to the vehicle in both rats groups at both dosages. When measuring DNA damage in Wistar rats, both Remembrance dosages had significantly less DNA damage compared to the vehicle group.

For genes affecting vascular readjustment, eNOS results in the production of NO which can increase vasodilatation. Upregulation of this gene is favorable for good vascular function and promoting angiogenesis. At 500 mg/kg/day, Remembrance treated Wistar rats significantly upregulated this gene compared to the vehicle.

In summary, mechanisms by which Remembrance may work to support brain health after an ischemic brain injury include increasing capillary density, upregulation of anti-apoptotic genes such as Bcl-xl and down regulation of proapoptotic genes such as AT₂, Bax, Fas and Bcl-Xs.

### Example 8

### Effect of Remembrance (Formulation A) on Memory: a Chronic Study

### Objectives:

The study aims to investigate the treatment effect of Remembrance in scopolamine-induced memory loss Sprague Dawley rat model compared with sham, vehicle, lingzhi (ganoderma lucidum) and ginkgo control groups.

### Methodology:

The Morris Water Maze Task test was used for testing the memory function of the Sprague Dawley rats weighing about 250 g. This task uses a round water pool (diameter: 2 meters) divided into four zones with a platform submerged beneath the surface in zone four. When placed in the maze, the rat's task is to find the hidden platform in the spatial acquisition test after being given scopolamine to induce memory impairment. Escape latency, distance swam and speed of swim are recorded during the spatial acquisition test.

A second probe trial test on day 6 was used to measure how much time each rat swam in each zone of the water pool once the submerged platform is removed. Two hours after the probe trial is completed the rates are decapitated and their brains are collected for further study measuring DNA damage, gene expression, and antioxidant assays. Molecular analysis also includes immunohistology testing and tunnel staining methods.

Each rat treatment group consists of 10 rats (See Table 20 d-b) for treatment groups and drug dosages.) Each treatment group is pretreated with their designated drugs from day 1-14. On days 1 and 7 pretraining of the rats in the water maze occurs. During pretraining days 1 and 7, each rat has two training sessions a day with 30 minutes rest in between each swim whereby they are given 120 sec. to find the submerged platform in zone four when placed in the maze randomly. If they are unable to find the platform after 120 sec then they are guided to the platform.

TABLE 20
Oral treatment
(Braintone = Remembrance = Formulation A)

| | | | Number |
|---|---|---|---|
| Group | Treatment | Dosage | of rats |
| Sham | | | 10 |
| Vehicle | Distilled water | | 10 |
| Braintone/ | Braintone/ | 500 mg/kg/day | 10 |
| Remembrance | Remembrance | | |
| Ginkgo | Ginkgo biloba extract | 100 mg/kg/day | 10 |
| Lingzhi | Winsor lingzhi extract | 500 mg/kg/day | 10 |

On days 15-19 the spatial acquisition test is carried out at the same time each day. Sixty minutes prior to the test, each rat is given their assigned drug according to their treatment group and 30 minutes prior they are given 1 mg/kg of scopolamine to induce memory impairment. (See Table 21 d-b) During each daily spatial acquisition test, each rat is given four consecutive trials of 120 sec. with 30 minutes rest in between to find the submerged platform when starting from a random point within the maze. Escape latency, distance swam and speed of swim are recorded during each trial.

TABLE 21
Treatment of experimental groups on testing group
(Braintone = Remembrance = Formulation A)

| Group | 60 mins prior to testing | 30 mins prior to testing |
|---|---|---|
| Sham | | |
| Vehicle | Distilled water | 1 mg/kg scopolamine |
| Braintone/ | 500 mg/kg Braintone/ | 1 mg/kg scopolamine |
| Remembrance | Remembrance | |
| Ginkgo | 100 mg/kg Ginkgo | 1 mg/kg scopolamine |
| | biloba extract | |
| Lingzhi | 500 mg/kg Winsor | 1 mg/kg scopolamine |
| | lingzhi extract | |

On day 20, after rats are given their assigned drug according to their treatment group the platform is removed from the maze and the probe trial is done whereby rats are given 120 sec to swim in the maze. Time spent and distance swam in each zone are recorded.

Two hours after the probe trial is complete, the rats are decapitated and brains are collected for further study measuring DNA damage, gene expression, and antioxidant assays. Molecular analysis also includes immunohistology testing and tunnel staining methods.

### Results:

### Spatial Acquisition Test:

As shown in FIG. 56, Remembrance-treated group (500 mg/kg/day) had the lowest mean escape latency compared to all other treatment groups on Day 3.

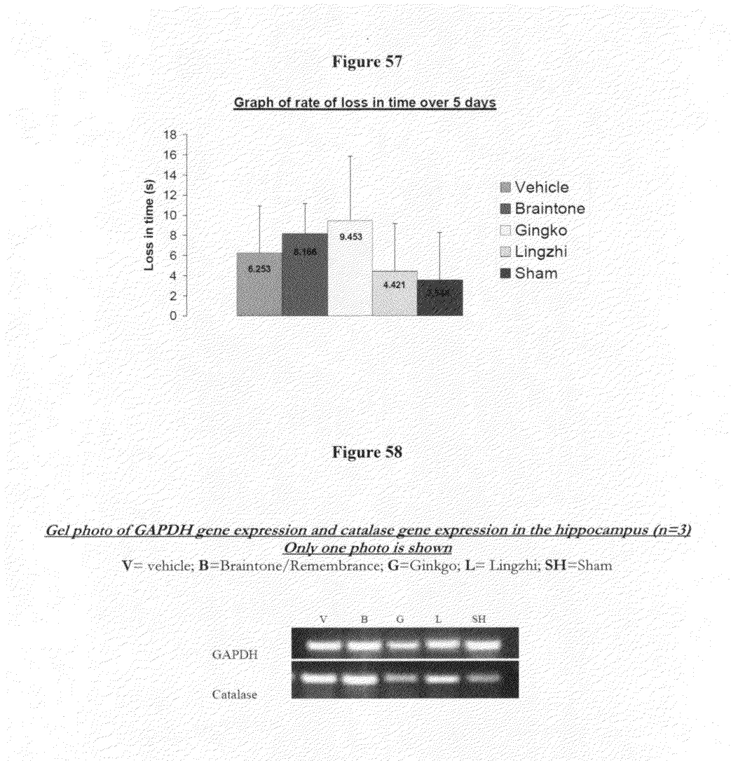

As shown in FIG. 57, The escape latency decreased over the 5 days of spatial acquisition test. All groups exhibited a learning effect.

Gene Expression—GAPD was used as an internal standard and standardized every time before PCR was done for each target gene. (n=3)

### Anti-Oxidant Genes

Scopolamine-induced memory loss Sprague Dawley rats (vehicle group) potentially have an increase of oxidative stress in brain cells which generally reduces the antioxidant gene expressions and antioxidant enzymatic activities. (Example: Catalase, GST, SOD, GPx)

As shown in FIG. 58, Remembrance-treatment group (500 mg/kg/day) had the strongest catalase gene expression in the hippocampus with fold difference of about 1.4.

Remembrance can generally up-regulate the catalase gene expression in defense of the scopolamine memory-loss challenge.

### GST

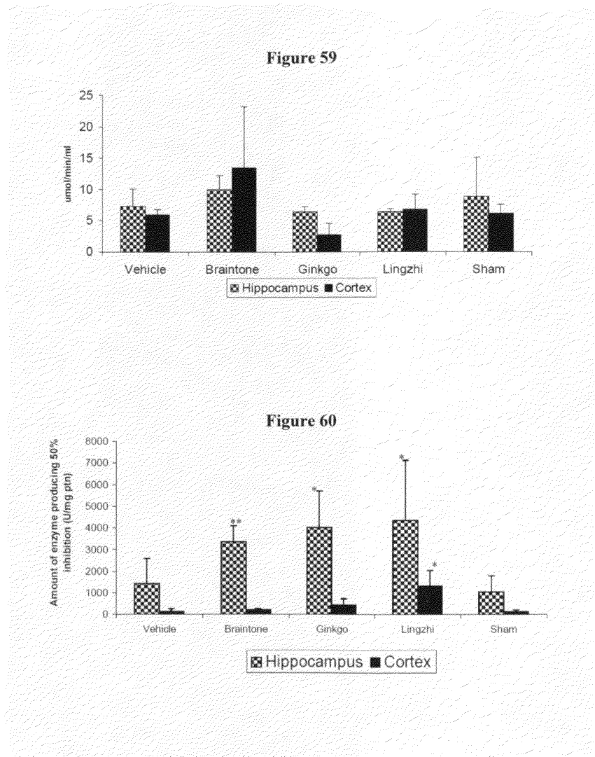

As shown in FIG. 59, Remembrance-treated group had the highest GST activity in both the hippocampus and the cortex.

### SOD

As shown in FIG. 60, The Remembrance-treated group exhibited significantly higher SOD activity in the hippocampus (n=3, p<0.01) compared to vehicle group. It is a higher significance then either gingko or lingzhi alone.

### DNA Damage

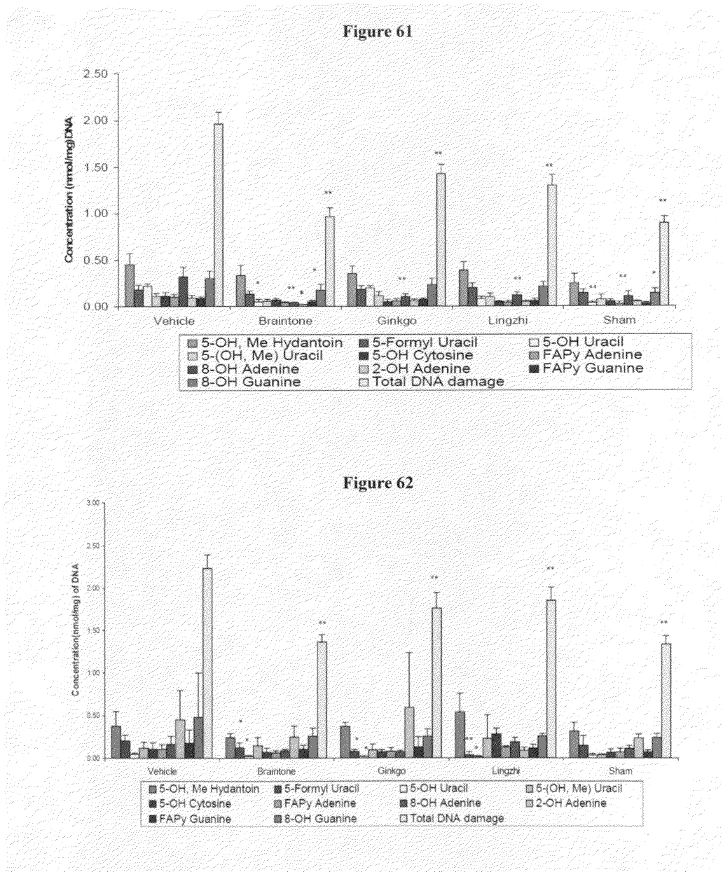

As shown in FIG. 61, Remembrance-treated group showed significantly less damaged bases in DNA extracts from blood compared to the vehicle group, particularly for 5-OH uracil (n=7, p<0.05), 8-OH adenine (n=7, p<0.01), 2-OH adenine (n=7, p<0.05) and 8-OH guanine (n=7, p<0.05). Remembrance was also able to significantly reduce extensive range of damaged DNA compared with vehicle group (n=7, p<0.01).

As shown in FIG. 62, Remembrance had significant reduction of total brain tissue DNA damage (compared with vehicle group), particularly for 5-OH uracil and 5-Formyl uracil (n=7, p<0.05).

### Discussion:

The brain is the major part of central nervous system; it controls many important activities like thoughts, memory, cognitive, learning ability and other vital physiological activities such as heart rate and respiration. Since brain cells perform all activities, their health is very important in maintaining our body's functions. However, brain cells are sensitive cells and susceptible to damage when there is poor circulation supplying oxygen and nutrients.

The results show that rats treated with Remembrance (500 mg/kg/day) for 20 days had similar performance on the water maze task compared to other groups in the special acquisition test and slightly better performance on the probe test with regards to speed and time in zone four compared with other treatment groups suggesting possible enhanced learning ability resulting from treatment with Remembrance. This supports the results found in EXAMPLE 1 that Remembrance may better preserve neurological function and blood circulation compared to ginkgo alone. One theory for better performance on the water maze task compared to other groups may be attributed to Remembrance's ability to promote blood circulation and delivery of nutrients to the brain in order to ensure the continued health of neuron cells, thereby maintaining brain cognitive and memory functions.

In this study, the gene expression and enzymatic activity of antioxidant enzymes: catalase, GST, GPx and SOD were investigated in the hippocampus and cortex. It has been reported that scopolamine potentially increases oxidative stress in the cells by reducing the amount of antioxidant enzymes in the brain. Remembrance can help to increase the gene expression of antioxidant enzyme (e.g. Catalase & SOD) and the enzymatic activities of GST and significantly SOD (p<0.01) in the hippocampus compared to the vehicle. An increase of antioxidant enzymatic activity may explain why Remembrance exhibited significantly less DNA damage in the blood and cerebral cortex compared with the vehicle group (See section on DNA damage). With less DNA damage in the cortex, there is likely less apoptosis which is an important factor for neuro-protection and memory health.

Additionally, the Remembrance-treated group had a significantly higher c-Fos expression (p<0.05) than the vehicle group in the hippocampus as well as a trend of increase in c-Jun expression. This implies that Remembrance may bring about increased memory consolidation by subsequent downstream transcription of proteins involved in synaptic plasticity.

### Example 9

### Cardioprotective Effects of Remembrance (Formulation A) in Wistar Rats

Four different doses of Remembrance (i.e. saline—0 mg/kg/day, 100 mg/kg/day, 250 mg/kg/day and 500 mg/kg/day) were administered in Wistar rats (n=8 each group) once daily for 7 days before the induction of myocardial infarction (MI). MI was induced by permanent ligation of left desending coronary artery (LDA). Successful ligation of LDA was verified visually by the change in color of the ischemic area. Infract size (Infarction) can be found in all MI Wistar rats. The size of the infarct area is calculated by Scion Image software (CA, USA).

Same administration continued for another 7 days after the induction of MI. Mortality rate and infract size in each group was compared.

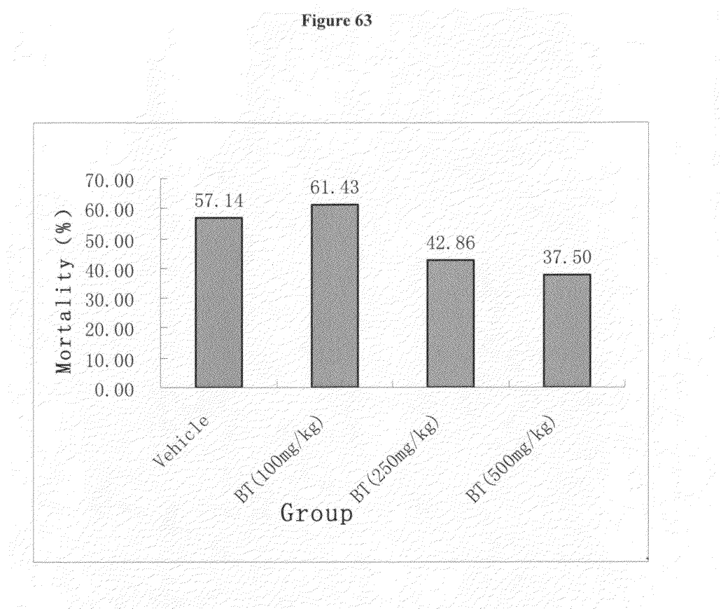

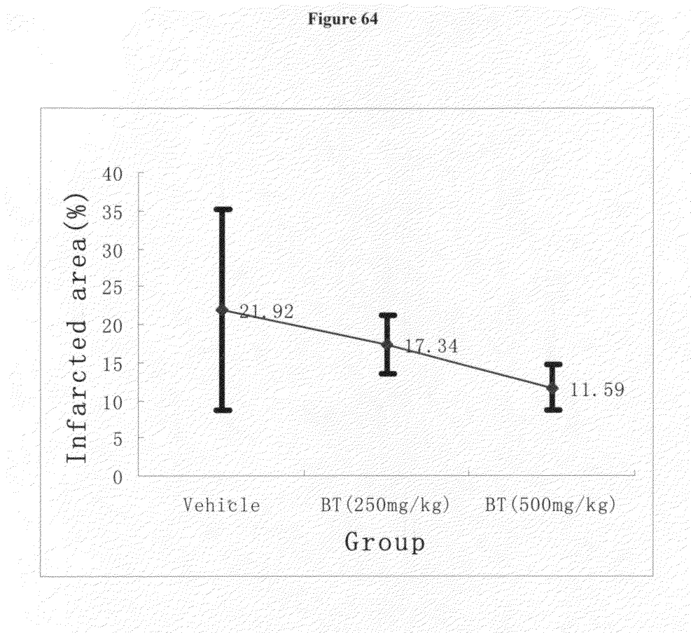

As shown in FIGS. 63 and 64, Remembrance administration before and after the MI induction can lower the death rate and infarct size development comparing to the control (saline group) which indicates its cardioprotective effects.

1. A method for preventing or reducing the risk of developing cardiovascular disease, comprising: administering a composition comprising Rhodiola rosea (root), Ginkgo biloba (leaf), Panax notoginseng (root), and Ligusticum chuanxiong (rhizome) to a mammal in need of such prevention or at risk of developing cardiovascular disease, wherein the concentration of Rhodiola rosea is about 5-95% w/w based on the total weight of the composition.

2. The method of claim 1, wherein the composition is administered to a human at a dose of about 1-600 mg/kg/day, about 10-500 mg/kg/day, about 20-300 mg/kg/day, about 50-200 mg/kg/day, about 7-40 mg/kg/day, or about 8-25 mg/kg/day.

3. The method of claim 1, wherein the composition is orally administered to a human at a dose of about 10-100 mg/kg, 20-60 mg/kg, or 30-50 mg/kg once or twice a day.

4. The method of claim 1, wherein the composition is orally administered to a human at a dose of about 10-200 mg/kg, 20-100 mg/kg, or 40-80 mg/kg once or twice a day.

5. The method of claim 1, wherein the cardiovascular disease is myocardial infarction.

6. The method of claim 1, wherein the cardiovascular disease is arteriosclerosis or atherosclerosis.

7. The method of claim 1, wherein the concentration of Ginkgo biloba is about 5-50% w/w based on the total weight of the composition.

8. The method of claim 1, wherein the concentration of Panax notoginseng is about 5-50%.

9. The method of claim 1, wherein the concentration of Ligusticum chuanxiong is about 1-50% w/w based on the total weight of the composition.

10. The method of claim 1, wherein the composition is a combination of extracts of Rhodiola rosea at about 50-85%; Ginkgo biloba at about 10-30%; Panax notoginseng at about 10-20%; and Ligusticum chuanxiong at about 3-8% w/w based on the total weight of the composition.

11. The method of claim 1, wherein the composition is a combination of extracts of Rhodiola rosea (root) at about 75%; Ginkgo biloba (leave) at about 10%; Panax notoginseng at about 10%; and Ligusticum chuanxiong (rhizoma) at about 5% w/w based on the total weight of the composition.

12. The method of claim 1, wherein the composition is a combination of extracts of Rhodiola rosea (root) at about 50%; Ginkgo biloba (leave) at about 35%; Panax notoginseng at about 10%; and Ligusticum chuanxiong (rhizoma) at about 5% w/w based on the total weight of the composition.

13. A method for promoting blood circulation in the body of a mammal comprising: administering a composition comprising Rhodiola rosea (root), Ginkgo biloba (leaf), Panax notoginseng (root), and Ligusticum chuanxiong (rhizome) to a mammal in need of promotion of blood circulation in the body.

14. The method of claim 13, wherein the composition is administered to a human at a dose of about 1-600 mg/kg/day, about 10-500 mg/kg/day, about 20-300 mg/kg/day, about 50-200 mg/kg/day, about 7-40 mg/kg/day, or about 8-25 mg/kg/day.

15. The method of claim 13, wherein the composition is orally administered to a human at a dose of about 10-100 mg/kg, 20-60 mg/kg, or 30-50 mg/kg once or twice a day.

16. The method of claim 13, wherein the composition is orally administered to a human at a dose of about 10-200 mg/kg, 20-100 mg/kg, or 40-80 mg/kg once or twice a day.

17. A method for promoting brain health in a mammal, comprising: administering a composition comprising Rhodiola rosea (root), Ginkgo biloba (leaf), Panax notoginseng (root), and Ligusticum chuanxiong (rhizome) to a mammal in need of promotion of brain health, wherein the concentration of Rhodiola rosea is about 5-95% w/w based on the total weight of the composition.

18. The method of claim 17, wherein the composition is administered to a human at a dose of about 1-600 mg/kg/day, about 10-500 mg/kg/day, about 20-300 mg/kg/day, about 50-200 mg/kg/day, about 7-40 mg/kg/day, or about 8-25 mg/kg/day.

19. The method of claim 17, wherein the composition is orally administered to a human at a dose of about 10-100 mg/kg, 20-60 mg/kg, or 30-50 mg/kg once or twice a day.

20. The method of claim 17, wherein the composition is orally administered to a human at a dose of about 10-200 mg/kg, 20-100 mg/kg, or 40-80 mg/kg once or twice a day.

21. A method for restoring or improving memory in a mammal, comprising: administering a composition comprising Rhodiola rosea (root), Ginkgo biloba (leaf), Panax notoginseng (root), and Ligusticum chuanxiong (rhizome) to a mammal in need of restoration or improvement of memory, wherein the concentration of Rhodiola rosea is about 5-95% w/w based on the total weight of the composition.

22. A method for preventing or reducing the risk of developing a brain disorder, comprising: administering a composition comprising Rhodiola rosea (root), Ginkgo biloba (leaf), Panax notoginseng (root), and Ligusticum chuanxiong (rhizome) to a mammal in need of such prevention or at risk of developing a brain disorder, wherein the concentration of Rhodiola rosea is about 5-95% w/w based on the total weight of the composition.

23. The method of claim 22, wherein the brain disorder is ischemic stroke, a neurodegenerative disease, or a vascular disorder.

24. The method of claim 22, wherein the neurodegenerative disease is selected from the group consisting of Huntington's disease, Alzheimer's disease, and amyotrophic lateral sclerosis.

25. The method of claim 22, wherein the vascular disorder is selected from the group consisting of arteriosclerosis, congestive heart failure, hypertension, cardiovascular diseases, cerebrovascular disease, renovascular disease, mesenteric vascular disease, pulmonary vascular disease, ocular vascular disease, peripheral vascular disease, and peripheral ischemic disease.

26. The method of claim 22, wherein the composition is administered to a human at a dose of about 1-600 mg/kg/day, about 10-500 mg/kg/day, about 20-300 mg/kg/day, about 50-200 mg/kg/day, about 7-40 mg/kg/day, or about 8-25 mg/kg/day.

27. The method of claim 22, wherein the composition is administered to a human at a dose of about 10-100 mg/kg, 20-60 mg/kg, or 30-50 mg/kg once or twice a day.

28. The method of claim 22, wherein the composition is administered to a human at a dose of about 10-200 mg/kg, 20-100 mg/kg, or 40-80 mg/kg once or twice a day.


SEQUENCE LISTING (10 sequences)

SEQ ID NO: 1 - DNA, 20 bp, Artificial Sequence
  Description of Artificial Sequence Synthetic primer
catggtctacatgttccagt

SEQ ID NO: 2 - DNA, 20 bp, Artificial Sequence
  Description of Artificial Sequence Synthetic primer
ggctaagcagttggtggtgc

SEQ ID NO: 3 - DNA, 20 bp, Artificial Sequence
  Description of Artificial Sequence Synthetic primer
ttgctgccaccagcagaaac

SEQ ID NO: 4 - DNA, 24 bp, Artificial Sequence
  Description of Artificial Sequence Synthetic primer
gtgtgggcctccaaaccattgcta

SEQ ID NO: 5 - DNA, 22 bp, Artificial Sequence
  Description of Artificial Sequence Synthetic primer
gcagggaggatggctggggaga

SEQ ID NO: 6 - DNA, 22 bp, Artificial Sequence
  Description of Artificial Sequence Synthetic primer
tccagacaagcagccgctcacg

SEQ ID NO: 7 - DNA, 21 bp, Artificial Sequence
  Description of Artificial Sequence Synthetic primer
aacatgagaacatcctgtgcc

SEQ ID NO: 8 - DNA, 21 bp, Artificial Sequence
  Description of Artificial Sequence Synthetic primer
tccctgctcatgatgtctacc

SEQ ID NO: 9 - DNA, 21 bp, Artificial Sequence
  Description of Artificial Sequence Synthetic primer
aaaatgtctcagagcaaccgg

SEQ ID NO: 10 - DNA, 21 bp, Artificial Sequence
  Description of Artificial Sequence Synthetic primer
tcacttccgactgaagagtga